(19) United States
(12) Patent Application Publication
(10) Pub. No.: US 2004/0117863 A1
(43) Pub. Date: Jun. 17, 2004

(54) Transgenically produced fusion proteins

(76) Inventors: Michael D. Edge, Macclesfield (GB); Dan Pollock, Medway, MA (US); Yann Echelard, Jamaica Plain, MA (US); Harry M. Meade, Newton, MA (US); Susanna M. Rybak, Frederick, MD (US)

Correspondence Address: GTC BIOTHERAPEUTICS, INC., 175 CROSSING BOULEVARD, SUITE 410, FRAMINGHAM, MA, 01702, US

(21) Appl. No.: 10/608,710

(22) Filed: Jun. 27, 2003

Related U.S. Application Data
(62) Division of application No. 09/398,610, filed on Sep. 17, 1999.
(60) Provisional application No. 60/101,083, filed on Sep. 18, 1998.

(51) Int. Cl. A01K 67/027

(52) U.S. Cl. 800/7

(57) ABSTRACT

A method of making a transgenic fusion protein. The method inlcudes providing a transgenic animal which includes a transgene which provides for the expression of the fusion protein; allowing the transgene to be expressed; and, recovering the fusion protein, from the milk of the transgenic animal.

### RELATED APPLICATIONS

This application claims the benefit of a previously filed Provisional Application No. 60/101,083 filed Sep. 18, 1998, which is hereby incorporated by reference.

### FUNDING

Work described herein has been funded in part with Federal funds from the National Cancer Institute, National Institutes of Health, under Contract No. NO1-CO-60000.

### FIELD OF THE INVENTION

The invention relates to transgenically produced fusion proteins (e.g., immunoglobulin-enzyme fusion proteins), nucleic acids which encode fusion proteins, and methods of making and using fusion proteins and nucleic acids.

### BACKGROUND OF THE INVENTION

A growing number of recombinant proteins are being developed for therapeutic and diagnostic applications. However, many of these proteins may be difficult or expensive to produce in a functional form and/or in the required quantities using conventional methods. Conventional methods involve inserting the gene responsible for the production of a particular protein into host cells such as bacteria, yeast, or mammalian cells, e.g., COS cells, and then growing the cells in culture media. The cultured cells then synthesize the desired protein. Traditional bacteria or yeast systems may be unable to produce many complex proteins in a functional form. While mammalian cells can reproduce complex proteins, they are generally difficult and expensive to grow, and often produce only mg/L quantities of protein. The limitations using bacterial, yeast or mammalian systems are particularly applicable to complex proteins, such as immunoglobulin-enzyme fusion proteins, that require proper post-translational modifications and assembly to be in functional form.

### SUMMARY OF THE INVENTION

In general, the invention features, a method of making a transgenic fusion protein, e.g., an immunoglobulin-enzyme fusion protein. The method includes providing a transgenic animal, e.g., goat or a cow, which includes a transgene which provides for the expression of the fusion protein, e.g., an immunoglobulin-enzyme fusion protein; allowing the transgene to be expressed; and, preferably, recovering the fusion protein, from the milk of the transgenic animal. (Although the embodiment described relates to expression in milk other promoters, e.g, other tissue specific promoters, e.g., muscle, hair, urine, blood, or eggs specific promoters can be used to produce fusion proteins in other tissues or products.)

In a preferred embodiment the transgene includes a first member fused to a second member. The first member can include the subunit of a targeting molecule, e.g., an Ig subunit, e.g., a subunit of an Ig specific for a tumor antigen (e.g., carcinoembryonic antigen (CEA), a transferrin receptor, TAG-72, an epidermal growth factor receptor). The second member can be: an enzyme; a polypeptide other than an Ig subunit, or fragment thereof; an Rnase, e.g., RnaseA, e.g., angiogenin; or carboxypeptidase B enzyme.

In preferred embodiments, the transgenic fusion protein is made in a mammary gland of the transgenic mammal, e.g., a ruminant, e.g., a goat or a cow.

In preferred embodiments, the transgenic fusion protein is secreted into the milk of the transgenic mammal, e.g., a ruminant, e.g., a dairy animal, e.g., a goat or a cow.

In preferred embodiments, the transgenic fusion protein is secreted into the milk of a transgenic mammal at concentrations of at least about 0.1 mg/ml, 0.5 mg/ml, 1.0 mg/ml, 1.5 mg/ml, 2 mg/ml, 3 mg/ml, 5 mg/ml or higher.

In preferred embodiments, the transgenic fusion protein is made under the control of a mammary gland specific promoter, e.g., a milk specific promoter, e.g., a milk serum protein or casein promoter. The milk specific promoter can be a casein promoter, beta lactoglobulin promoter, whey acid protein promoter, or lactalbumin promoter. Preferably, the promoter is a goat β casein promoter.

In preferred embodiments, the transgenic fusion has the formula: R1-L-R2; R2-L-R1; R2-R1; or R1-R2, wherein R1 is an immunoglobulin moiety, L is a peptide linker and R2 is an enzyme moiety. Preferably, R1 and R2 are covalently linked, e.g., directly fused or linked via a peptide linker.

In preferred embodiments, the transgenic fusion protein further includes:

a signal sequence which directs the secretion of the fusion protein, e.g., a signal from a secreted protein (e.g., a signal from a protein secreted into milk, or an immunoglobulin signal); and

(optionally) a sequence which encodes a sufficient portion of the amino terminal coding region of a secreted protein, e.g., a protein secreted into milk, or an immunoglobulin, to allow secretion, e.g., in the milk of a transgenic mammal, of the fusion protein.

In preferred embodiments, the fusion protein includes a monoclonal antibody subunit, e.g., a human, murine (e.g., mouse) monoclonal antibody subunit, or a fragment thereof, e.g., an antigen binding fragment thereof The monoclonal antibody subunit or antigen binding fragment thereof can be a single chain polypeptide, a dimer of a heavy chain and a light chain, or a tetramer of two heavy and two light chains. Preferably, the monoclonal antibody is a human antibody or a fragment thereof, e.g., an antigen binding fragment thereof. For example, the human antibody can be produced by a hybridoma which includes a B cell obtained from a transgenic non-human animal, e.g., a transgenic mouse, having a genome comprising a human heavy chain transgene and a light chain transgene fused to an immortalized cell. The antibodies can be of the various isotypes, including: IgG (e.g., IgG1, IgG2, IgG3, IgG4), IgM, IgA1, IgA2, IgA.sub.sec, IgD, of IgE. Preferably, the antibody is an IgG isotype. The antibodies can be full-length (e.g., an IgG1 or IgG4 antibody) or can include only an antigen-binding portion (e.g., a Fab, F(ab′)₂, Fv or a single chain Fv fragment).

In preferred embodiments, the immunoglobulin subunit of a fusion protein is a recombinant antibody, e.g., a chimeric or a humanized antibody, subunit or an antigen binding fragment thereof, e.g., has a variable region, or at least a complementarity determining region (CDR), derived from a non-human antibody (e.g., murine) with the remaining portion(s) are human in origin.

In preferred embodiments, the immunoglobulin subunit of the fusion protein is monovalent (e.g., it included one pair of heavy and light chains, or antigen binding portions thereof). In other embodiments, the fusion protein is divalent antibody (e.g., it included two pairs of heavy and light chains, or antigen binding portions thereof).

In preferred embodiments, the transgenic fusion protein includes an immunoglobulin heavy chain or a fragment thereof, e.g., an antigen binding fragment thereof. Preferably, the immunoglobulin heavy chain or fragment thereof (e.g., an antigen binding fragment thereof) is linked, e.g., linked via a peptide linker or is directly fused, to an enzyme. Preferably, the immunoglobulin heavy chain-enzyme fusion protein is capable of assembling into a functional complex, e.g., a di-, tri-, tetra-, or multi-meric complex having enzymatic activity.

In preferred embodiments, the transgenic fusion protein includes an immunoglobulin heavy chain or fragment thereof (e.g., an antigen binding fragment thereof), and a light chain or a fragment thereof (e.g., an antigen binding fragment thereof). Preferably, the immunoglobulin heavy chain is linked, e.g., linked via a peptide linker or directly fused, to an enzyme. Preferably, the immunoglobulin-enzyme fusion protein is capable of assembling into a functional complex, e.g., a di-, tri-, tetra-, or multi-meric complex having enzymatic activity.

In preferred embodiments, the enzyme of the fusion protein is an Rnase, e.g., RnaseA, e.g., angiogenin; or carboxypeptidase B enzyme. For diagnostic applications, the enzyme can be horseradish peroxidase.

In a preferred embodiment, the transgenic fusion protein includes a peptide linker and the peptide linker has one or more of the following characteristics: a) it allows for the rotation of the immunoglobulin protein and the enzyme protein relative to each other; b) it is resistant to digestion by proteases; c) it does not interact with the immunoglobulin or the enzyme; d) it allows the fusion protein to form a complex (e.g., a di-, tri-, tetra-, or multi-meric complex) that retains enzymatic activity; and e) it promotes folding and/or assembly of the fusion protein into an active complex.

In a preferred embodiment: the transgenic fusion protein includes a peptide linker and the peptide linker is 5 to 60, more preferably, 10 to 30, amino acids in length; the peptide linker is 20 amino acids in length; the peptide linker is 17 amino acids in length; each of the amino acids in the peptide linker is selected from the group consisting of Gly, Ser, Asn, Thr and Ala; the peptide linker includes a Gly-Ser element.

In a preferred embodiment, the transgenic fusion protein includes a peptide linker and the peptide linker includes a sequence having the formula (Ser-Gly-Gly-Gly-Gly)_{y} wherein y is 1, 2, 3, 4, 5, 6, 7, or 8. Preferably, the peptide linker includes a sequence having the formula (Ser-Gly-Gly-Gly-Gly)₃. Preferably, the peptide linker includes a sequence having the formula ((Ser-Gly-Gly-Gly-Gly)₃-Ser-Pro).

In preferred embodiments, the transgenic fusion protein assembles into a dimer, trimer, tetramer, or higher polymeric complex.

In preferred embodiments, the transgene encoding the transgenic fusion protein is a nucleic acid construct which includes:

(a) optionally, an insulator sequence;

(b) a promoter, e.g., a mammary epithelial specific promoter, e.g., a milk protein promoter;

(c) a nucleotide sequence which encodes a signal sequence which can direct the secretion of the fusion protein, e.g. a signal from a milk specific protein;

(d) optionally, a nucleotide sequence which encodes a sufficient portion of the amino terminal coding region of a secreted protein, e.g. a protein secreted into milk, to allow secretion, e.g., in the milk of a transgenic mammal, of the non-secreted protein;

(e) one or more nucleotide sequences which encode the fusion protein, e.g., an immunoglobulin-enzyme fusion protein, e.g., a protein as described herein; and

(f) optionally, a 3′ untranslated region from a mammalian gene, e.g., a mammary epithelial specific gene, (e.g., a milk protein gene).

In preferred embodiments, elements a (if present), b, c, d (if present), and f of the transgene are from the same gene; the elements a (if present), b, c, d (if present), and f of the transgene are from two or more genes. For example, the signal sequence, the promoter sequence and the 3′ untranslated sequence can be from a mammary epithelial specific gene, e.g., a milk serum protein or casein gene (e.g., a β casein gene). Preferably, the signal sequence, the promoter sequence and the 3′ untranslated sequence are from a goat β casein gene.

In preferred embodiments, the promoter of the transgene is a mammary epithelial specific promoter, e.g., a milk serum protein or casein promoter (e.g., a β casein promoter). The milk specific promoter can be a casein promoter, beta lactoglobulin promoter, whey acid protein promoter, or lactalbumin promoter. Preferably, the promoter is a goat β casein promoter.

In preferred embodiments, the signal sequence encoded by the transgene is an amino terminal sequence which directs the expression of the protein to the exterior of a cell, or into the cell membrane. Preferably, the signal sequence is from a protein which is secreted into the milk, e.g., the milk of the transgenic animal.

In preferred embodiments, the 3′ untranslated region of the transgene includes a polyadenylation site, and is obtained from a mammary epithelial specific gene, e.g., a milk serum protein gene or casein gene. The 3′ untranslated region can be obtained from a casein gene (e.g., a β casein gene), a beta lactoglobulin gene, whey acid protein gene, or lactalbumin gene. Preferably, the 3′ untranslated region is from a goat β casein gene.

In preferred embodiments, the transgene, e.g., the transgene as described herein, integrates into a germ cell and/or a somatic cell of the transgenic animal.

In another aspect, the invention features a nucleic acid construct, preferably, an isolated nucleic acid construct, which includes:

(a) optionally, an insulator sequence;

(b) a promoter, e.g., a mammary epithelial specific promoter, e.g., a milk protein promoter;

(c) a nucleotide sequence which encodes a signal sequence which can direct the secretion of the fusion protein, e.g. a signal sequence from a milk specific protein;

(d) optionally, a nucleotide sequence which encodes a sufficient portion of the amino terminal coding region of a secreted protein, e.g. a protein secreted into milk, to allow secretion, e.g., in the milk of a transgenic mammal, of the non-secreted protein;

(e) one or more nucleotide sequences which encode a fusion protein as described herein; and

(f) optionally, a 3′ untranslated region from a mammalian gene, e.g., a mammary epithelial specific gene, (e.g., a milk protein gene).

In preferred embodiments, the promoter is a mammary epithelial specific promoter, e.g., a milk serum protein or casein promoter (e.g., a β casein promoter). The milk specific promoter can be a casein promoter, beta lactoglobulin promoter, whey acid protein promoter, or lactalbumin promoter. Preferably, the promoter is a goat β casein promoter.

In preferred embodiments, the signal sequence is an amino terminal sequence which directs the expression of the protein to the exterior of a cell, or into the cell membrane. Preferably, the signal sequence is from a milk specific protein. Preferably, the signal sequence directs secretion of the encoded fusion protein into the milk of a transgenic animal, e.g., a transgenic mammal.

In preferred embodiments, the 3′ untranslated region includes a polyadenylation site, and is obtained from a mammalian gene, e.g., a mammary epithelial specific gene, e.g., a milk serum protein gene or casein gene. The 3′ untranslated region can be obtained from a casein gene (e.g., a β casein gene), a beta lactoglobulin gene, whey acid protein gene, or lactalbumin gene. Preferably, the 3′ untranslated region is from a goat β casein gene.

In another aspect, the invention features a host cell, e.g., an isolated host cell, which includes a nucleic acid of the invention (e.g., a transgene, e.g., a nucleic acid construct as described herein).

In another aspect, the invention features, a pharmaceutical or nutraceutical composition having an effective amount of fusion protein, e.g., an immunoglobulin-enzyme fusion protein as described herein, and a pharmaceutically acceptable carrier.

In a preferred embodiment, the composition includes milk.

In another aspect, the invention features, a transgenic animal which includes a transgene that encodes a fusion protein, e.g., a transgene which encodes an immunoglobulin-enzyme fusion protein described herein.

Preferred transgenic animals include: mammals; birds; reptiles; marsupials; and amphibians. Suitable mammals include: ruminants; ungulates; domesticated mammals; and dairy animals. Particularly preferred animals include: mice, goats, sheep, camels, rabbits, cows, pigs, horses, oxen, and llamas. Suitable birds include chickens, geese, and turkeys. Where the transgenic protein is secreted into the milk of a transgenic animal, the animal should be able to produce at least 1, and more preferably at least 10, or 100, liters of milk per year. Preferably, the transgenic animal is a ruminant, e.g., a goat, cow or sheep. Most preferably, the transgenic animal is a goat.

In preferred embodiments, the transgenic animal has germ cells and somatic cells containing a transgene that encodes a fusion protein, e.g, a fusion protein described herein.

In preferred embodiments, the fusion protein expressed in the transgenic animal is under the control of a mammary gland specific promoter, e.g., a milk specific promoter, e.g., a milk serum protein or casein promoter. The milk specific promoter can be a casein promoter, beta lactoglobulin promoter, whey acid protein promoter, or lactalbumin promoter. Preferably, the promoter is a goat P casein promoter.

In preferred embodiments, the transgenic animal is a mammal, and the immunoglobulin-enzyme fusion protein is secreted into the milk of the transgenic animal at concentrations of at least about 0.1 mg/ml, 0.5 mg/ml, 1.0 mg/ml, 1.5 mg/ml, 2 mg/ml, 3 mg/ml, 5 mg/ml or higher.

In another aspect, the invention features, a method of selectively killing or lysing an aberrant or diseased cell which expresses on its surface a target antigen. The method includes:

contacting said aberrant or diseased cell with a transgenically produced fusion protein, e.g., a transgenically produced immunoglobulin-enzyme fusion protein described herein, wherein the immunoglobulin of said fusion protein recognizes said target antigen,

The terms peptides, proteins, and polypeptides are used interchangeably herein.

A purified preparation, substantially pure preparation of a polypeptide, or an isolated polypeptide as used herein, means a polypeptide that has been separated from at least one other protein, lipid, or nucleic acid with which it occurs in the cell or organism which expresses it, e.g., from a protein, lipid, or nucleic acid in a transgenic animal or in a fluid, e.g., milk, or other substance, e.g., an egg, produced by a transgenic animal. The polypeptide is preferably separated from substances, e.g., antibodies or gel matrix, e.g., polyacrylamide, which are used to purify it. The polypeptide preferably constitutes at least 10, 20, 50 70, 80 or 95% dry weight of the purified preparation. Preferably, the preparation contains: sufficient polypeptide to allow protein sequencing; at least 1, 10, or 100 μg of the polypeptide; at least 1, 10, or 100 mg of the polypeptide.

A substantially pure nucleic acid, is a nucleic acid which is one or both of: not immediately contiguous with either one or both of the sequences, e.g., coding sequences, with which it is immediately contiguous (i.e., one at the 5′ end and one at the 3′ end) in the naturally-occurring genome of the organism from which the nucleic acid is derived; or which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure DNA also includes a recombinant DNA which is part of a hybrid gene encoding additional fusion protein sequence.

As used herein, the term transgene means a nucleic acid sequence (encoding, e.g., one or more fusion protein polypeptides), which is introduced into the genome of a transgenic organism. A transgene can include one or more transcriptional regulatory sequences and other nucleic acid, such as introns, that may be necessary for optimal expression and secretion of a nucleic acid encoding the fusion protein. A transgene can include an enhancer sequence. A fusion protein sequence can be operatively linked to a tissue specific promoter, e.g., mammary gland specific promoter sequence that results in the secretion of the protein in the milk of a transgenic mammal, a urine specific promoter, or an egg specific promoter.

As used herein, the term “transgenic cell” refers to a cell containing a transgene.

A “transgenic organism”, as used herein, refers to a transgenic animal or plant.

As used herein, a “transgenic animal” is a non-human animal in which one or more, and preferably essentially all, of the cells of the animal contain a transgene introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Mammals are defined herein as all animals, excluding humans, that have mammary glands and produce milk.

As used herein, a “dairy animal” refers to a milk producing non-human animal which is larger than a rodent. In preferred embodiments, the dairy animal produce large volumes of milk and have long lactating periods, e.g., cows or goats.

As used herein, the language “subject” includes human and non-human animals. The term “non-human animals” of the invention includes vertebrates, e.g., mammals and non-mammals, such as non-human primates, ruminants, birds, amphibians, reptiles and rodents, e.g., mice and rats. The term also includes rabbits.

As used herein, a “transgenic plant” is a plant, preferably a multi-celled or higher plant, in which one or more, and preferably essentially all, of the cells of the plant contain a transgene introduced by way of human intervention, such as by transgenic techniques known in the art.

As used herein, the term “plant” refers to either a whole plant, a plant part, a plant cell, or a group of plant cells. The class of plants which can be used in methods of the invention is generally as broad as the class of higher plants amenable to transformation techniques, including both monocotyledonous and dicotyledonous plants. It includes plants of a variety of ploidy levels, including polyploid, diploid and haploid.

As used herein, the term “nutraceutical,” refers to a food substance or part of a food, which includes a fusion protein. Nutraceuticals can provide medical or health benefits, including the prevention, treatment or cure of a disorder. The transgenic protein will often be present in the nutraceutical at concentration of at least 100 μg/kg, more preferably at least 1 mg/kg, most preferably at least 10 mg/kg. A nutraceutical can include the milk of a transgenic animal.

As used herein, the terms “immunoglobulin” and “antibody” refer to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g. a target antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et aL (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The term “monoclonal antibody” as used herein refers to an antibody molecule of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody”, as used herein, is intended to include all human antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human gernline VH and VL sequences, may not naturally exist within the human antibody germline repertoire in vivo.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

The terms “vector” or “construct”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated vectors.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Other features and advantages of the invention will be apparent from the following detailed description, and from the claims.

### DETAILED DESCRIPTION

The drawings are first described.

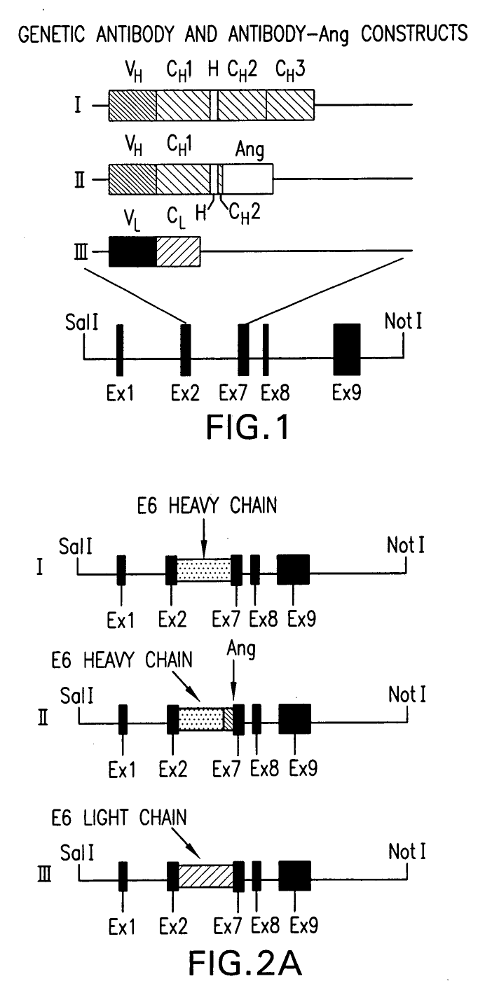

FIG. 1 is a schematic representation of the genetic antibody and antibody angiogenin fusion proteins.

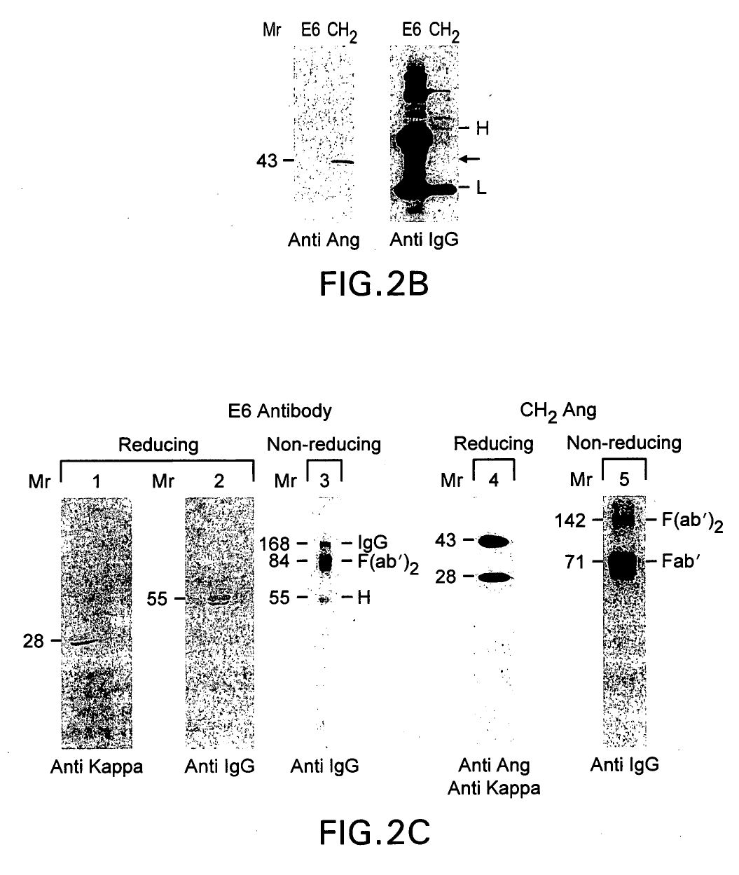

FIG. 2A is a schematic diagram of the structure of the transgenic expression vectors for the transferring receptor antibody (E6) and the angiogenin-enzyme fusion (CH2Ang). The following DNAs were fused between exons 2 and 7 of a modified goat β-casein gene (DiTullio et al., 1992) for expression in the mammary gland of mice; the heavy chain of the anti-human transferrin receptor monoclonal antibody, E6 (1); the same heavy chain fused at the CH2 domain to the 5′ end of the gene encoding angiogenin (Ang) as previously described (Rybak et al., 1992) (II); the light chain of the E6 antibody (III). Open boxes, heavy chain; crossed hatched boxes light chain: striped boxes, Ang.

FIG. 2B shows Western analysis using anti-angiogenin or anti-IgG antibodies under reducing conditions of milk collected form lactating females producing either E6 IgG antibody or CH2Ang fusion protein. 15 ul of milk diluted with an equal volume of PBS was applied to the gel.

FIG. 2C shows Western analysis of purified E6 antibody or CH2Ang under reducing or non-reducing conditions. The blots were analyzed with the indicated antibodies 0.3μ E6, lanes 1 and 2; 4 μg E6, lane 3;07 and 0.2 μg CH2Ang lanes 4 and 5, respectively.

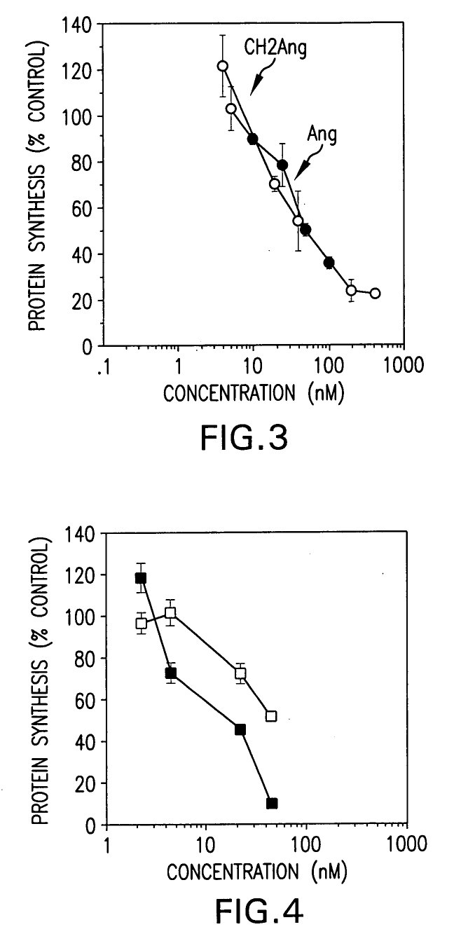

FIG. 3 is a graph depicting the effects of angiogenin or a fusion of angiogenin-antibody fusion (CH2Ang) on mRNA translation. Angiogenin or the fusion protein was added to a lysate mixture containing BMV mRNA and [³⁵S]methionine. Protein synthesis was determined by measuring the incorporation of label into newly synthesized protein as described in (Newton et al., 1996). Data from 2-3 experiments were pooled and plotted±SEM. The results are expressed as a percentage of a mock treated control reaction IC₅₀ is the concentration of Ang or the Ang fusion protein required to cause 50% inhibition of protein synthesis and was determined form the dose response curves. Solid circles, Ang;open circles, CH2 Ang.

FIG. 4 is a graph depicting a dose response curve showing the cytotoxic effect of the angiogenin-antibody fusion in cultured cells. In vivo toxicity of CH2Ang to SF539 and MDA-MB-231]^{mdr} cells as assessed by protein synthesis inhibition. Cytoxicity assays were performed by measuring the incorporation of [¹⁴C]leucine into cell proteins as described in Methods. the assays were conducted in the presence of serum and changed to leucine-and-serum-free medium prior to pulsing with [¹⁴C]leucine. IC₅₀ is the concentration of the angiogenin fusion proteins required to cause a 50% inhibition of protein synthase after 3 days and was determined directly from the dose response curves. The SEM is then when it is larger than the symbol. Solid symbols. SF539 human glioma cells; open symbols, MDA-MB-23]^{mdr1} human breast cancer cells.

The present invention provides, at least in part, transgenically produced fusion proteins. In one embodiment, the fusion protein includes an immunoglobulin subunit (e.g., an immunoglobulin heavy or light chain) fused to a toxin (e.g., a subunit of an enzyme). The immunogloblulin-enzyme fusion proteins described herein serve to target a cytotoxic agent (e.g. the enzyme) to an undesirable cell, e.g., a tumor cell. For example, the fusion proteins described in the Examples below, (i.e., an antibody against carcinoembryonic antigen (CEA) fused to an enzyme, e.g., RNAse A, or carboxypeptidase) can be used to target, to a tumor cell. After allowing sufficient time for the immunoglobulin-enzyme fusion to localize at the tumor site, a non-toxic prodrug can be administered. This prodrug is converted to a highly cytotoxic drug by the action of the targeted enzyme localized at the tumor site, permitting to achieve therapeutic levels of the drug without unacceptable toxicity for the patients.

### Production of Immunoglobulins

A monoclonal antibody against a target antigen, e.g., a cell surface protein (e.g., receptor) on a cell can be produced by a variety of techniques, including conventional monoclonal antibody methodology e.g., the standard somatic cell hybridization technique of Kohler and Milstein, Nature 256: 495 (1975). Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than the mouse system. Splenocytes from these transgernic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, Biotechniques 11:152-156). A similar strategy can also been used to amplify human heavy and light chain variable regions from human antibodies (Larrick et al., 1991, Methods: Companion to Methods in Enzymology 2:106-110).

In an illustrative embodiment, RNA is isolated from B lymphocytes, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combinantion, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

Once displayed on the surface of a display package (e.g., filamentous phage), the antibody library is screened with the target antigen, or peptide fragment thereof, to identify and isolate packages that express an antibody having specificity for the target antigen. Nucleic acid encoding the selected antibody can be recovered from the display package (e.g., from the phage genome) and subcloned into other expression vectors by standard recombinant DNA techniques.

Specific antibody molecules with high affinities for a surface protein can be made according to methods known to those in the art, e.g, methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies.

In particular, the Fv binding surface of a particular antibody molecule interacts with its target ligand according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence information, by a modeling procedure using previously determined three-dimensional structures from other antibodies obtained from NMR studies or crytallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol.51, op. cit., pp 1-15.

In one embodiment, a variegated peptide library is expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are described above.

Other techniques include affinity chromatography with an appropriate “receptor”, e.g., a target antigen, followed by identification of the isolated binding agents or ligands by conventional techniques (e.g., mass spectrometry and NMR). Preferably, the soluble receptor is conjugated to a label (e.g., fluorophores, calorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library (see, e.g., W. C. Still et al., International Application WO 94/08051). In general, this method features the use of inert but readily detectable tags, that are attached to the solid support or to the compounds. When an active compound is detected, the identity of the compound is determined by identification of the unique accompanying tag. This tagging method permits the synthesis of large libraries of compounds which can be identified at very low levels among to total set of all compounds in the library.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the hinge region, thus generating a monovalent antibody. Any modification is within the scope of the invention so long as the antibody has at least one antigen binding region specific.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method, which is capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or subsituted are referred to herein as modified antibodies or altered antibodies.

### Target Antigens

In preferred embodiments, a component of the fusion proteins of the present invention is a targeting agent, e.g., a polypeptide having a high affinity for a target, e.g., an antibody, a ligand, or an enzyme. Accordingly, the fusion proteins of the invention can be used to selectively direct (e.g., localize) the second component of the fusion protein to the vicinity of an undesirable cell.

For example, the first component can be an immunoglobulin that interacts with (e.g., binds to a target antigen). In certain embodiments, the target antigen is present on the surface of a cell, e.g., an aberrant cell such a hyperproliferative cell (e.g., a cancer cell). Exemplary target antigens include carcinoembryonic antigen (CEA), TAG-72, her-2/neu, epidermal growth factor receptor, transferrin receptor, among others. Preferably, the target antigen is carcinoembryonic antigen.

As used herein, “target cell” shall mean any undesirable cell in a subject (e.g., a human or animal) that can be targeted by a fusion protein of the invention. Exemplary target cells include tumor cells, such as carcinoma or adenocarcinoma-derived cells (e.g., colon, breast, prostate, ovarian and endometrial cancer cells) (Thor, A. et al. (1997) Cancer Res 46: 3118; Soisson A. P. et al. (1989) Am. J. Obstet. Gynecol.:1258-63). The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, ovarian carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

### Production of Fusion Proteins

The components of the fusion protein can be linked to each other, preferably via a linker sequence. The linker sequence should separate the first and second members of the fusion protein by a distance sufficient to ensure that each member properly folds into its secondary and tertiary structures. Preferred linker sequences (1) should adopt a flexible extended conformation, (2) should not exhibit a propensity for developing an ordered secondary structure which could interact with the functional first and second component, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence.

A linker sequence length of 20 amino acids can be used to provide a suitable separation of functional protein domains, although longer or shorter linker sequences may also be used. The length of the linker sequence separating the first and second components can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. Amino acid sequences useful as linkers of the first and second member include, but are not limited to, (SerGly4)_{y} wherein y is greater than or equal to 8, or Gly₄SerGly₅Ser. A preferred linker sequence has the formula (SerGly₄)₄. Another preferred linker has the sequence ((Ser-Ser-Ser-Ser-Gly)₃-Ser-Pro).

The first and second components can be directly fused without a linker sequence. Linker sequences are unnecessary where the proteins being fused have non-essential N-or C-termnmal amino acid regions which can be used to separate the functional domains and prevent steric interference. In preferred embodiments, the C-terminus of first member can be directly fused to the N-terminus of second, or viceversa.

### Recombinant Production

A fusion protein of the invention can be prepared with standard recombinant DNA techniques using a nucleic acid molecule encoding the fusion protein. A nucleotide sequence encoding a fusion protein can be synthesized by standard DNA synthesis methods.

A nucleic acid encoding a fusion protein can be introduced into a host cell, e.g., a cell of a primary or immortalized cell line. The recombinant cells can be used to produce the fusion protein. A nucleic acid encoding a fusion protein can be introduced into a host cell, e.g., by homologous recombination. In most cases, a nucleic acid encoding the fusion protein is incorporated into a recombinant expression vector.

The nucleotide sequence encoding a fusion protein can be operatively linked to one or more regulatory sequences, selected on the basis of the host cells to be used for expression. The term “operably linked” means that the sequences encoding the fusion protein compound are linked to the regulatory sequence(s) in a manner that allows for expression of the fusion protein. The term “regulatory sequence” refers to promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the content of which are incorporated herein by reference. Regulatory sequences include those that direct constitiitive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of fusion protein desired, and the like. The fusion protein expression vectors can be introduced into host cells to thereby produce fusion proteins encoded by nucleic acids.

Recombinant expression vectors can be designed for expression of fusion proteins in prokaryotic or eukaryotic cells. For example, fusion proteins can be expressed in bacterial cells such as E. coli, insect cells (e.g., in the baculovirus expression system), yeast cells or mammalian cells. Some suitable host cells are discussed further in Goeddel, Gene Expression Technology: Methods in Enzymology 185, Academic Press, San Diego, Calif. (1990). Examples of vectors for expression in yeast S. cerevisiae include pYepSec1 (Baldari et al., (1987) EMBO J. 6:229-234), pMFa (Kujan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of fusion proteins in cultured insect cells (e.g., Sf9 cells) include the pAc series (Smith et al., (1983) Mol. Cell. Biol. 3:2156-2165) and the pVL series (Lucklow, V. A., and Summers, M. D., (1989) Virology 170:31-39).

Examples of mammalian expression vectors include pCDM8 (Seed, B., (1987) Nature 329:840) and pMT2PC (Kaufinan et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the fusion protein from a host cell, in particular mammalian host cells, the recombinant expression vector can encode a signal sequence operatively linked to sequences encoding the amino-terminus of the fusion protein such that upon expression, the fusion protein is synthesized with the signal sequence fused to its amino terminus. This signal sequence directs the fusion protein into the secretory pathway of the cell and is then cleaved, allowing for release of the mature fusion protein (i.e., the fusion protein without the signal sequence) from the host cell. Use of a signal sequence to facilitate secretion of proteins or peptides from mammalian host cells is known in the art.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

Often only a small fraction of mammalian cells integrate the foreign DNA into their genome. In order to identify and select these integrants, a gene that encodes a selectable marker (e.g., resistance to antibiotics) can be introduced into the host cells along with the gene encoding the fusion protein. Preferred selectable markers include those that confer resistance to drugs, such as G418, hygromycin and methotrexate. Nucleic acid encoding a selectable marker can be introduced into a host cell on the same vector as that encoding the fusion protein or can be introduced on a separate vector. Cells stably transfected with the introduced nucleic acid can be identified by drug selection (e.g., cells that have incorporated the selectable marker gene will survive, while the other cells die).

A recombinant expression vector can be transcribed and translated in vitro, for example using T7 promoter regulatory sequences and T7 polymerase.

### Transgenic Mammals

Methods for generating non-human transgenic animals are described herein. DNA constructs can be introduced into the germ line of a mammal to make a transgenic mammal. For example, one or several copies of the construct can be incorporated into the genome of a mammalian embryo by standard transgenic techniques.

It is often desirable to express the transgenic protein in the milk of a transgenic mammal. Mammals that produce large volumes of milk and have long lactating periods are preferred. Preferred mammals are ruminants, e.g., cows, sheep, camels or goats, e.g., goats of Swiss origin, e.g., the Alpine, Saanen and Toggenburg breed goats. Other preferred animals include oxen, rabbits and pigs.

In an exemplary embodiment, a transgenic non-human animal is produced by introducing a transgene into the germline of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the fusion protein transgene into the embryo can be accomplished by any of a variety of means known in the art such as microinjection, electroporation, or lipofection. For example, a fusion protein transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct by Southern blot analysis of a segment of tissue. An embryo having one or more copies of the exogenous cloned construct stably integrated into the genome can be used to establish a permanent transgenic mammal line carrying the transgenically added construct.

Litters of transgenically altered mammals can be assayed after birth for the incorporation of the construct into the genome of the offspring. This can be done by hybridizing a probe corresponding to the DNA sequence coding for the fusion protein or a segment thereof onto chromosomal material from the progeny. Those mammalian progeny found to contain at least one copy of the construct in their genome are grown to maturity. The female species of these progeny will produce the desired protein in or along with their milk. The transgenic mammals can be bred to produce other transgenic progeny useful in producing the desired proteins in their milk.

Transgenic females may be tested for protein secretion into milk, using an art-known assay technique, e.g., a Western blot or enzymatic assay.

### Other Transgenic Animals

Fusion protein can be expressed from a variety of transgenic animals. A protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. A protocol for the production of a transgenic mouse can be found in U.S. Pat. No. 5,530,177. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic sheep can be found in Transgenic Animal Technology, A Handbook, 1994, ed., Carl A. Pinkert, Academic Press, Inc. A protocol for the production of a transgenic rabbit can be found in Hammer et al., Nature 315:680-683, 1985 and Taylor and Fan, Frontiers in Bioscience 2: d298-308, 1997.

### Production of Transgenic Protein in the Milk of a Transgenic Animal

### Milk Specific Promoters

Useful transcriptional promoters are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gorton et al. (1987) Bio/Technology 5: 1183-1187), and lactalbumin (Soulier et al., (1992) FEBS Letts. 297: 13). The alpha, beta, gamma or kappa casein gene promoter of any mammalian species can be used to provide mammary expression; a preferred promoter is the goat beta casein gene promoter (DiTullio, (1992) Bio/Technology 10:74-77). Milk-specific protein promoter or the promoters that are specifically activated in mammary tissue can be isolated from cDNA or genomic sequences. Preferably, they are genomic in origin.

DNA sequence information is available for mammary gland specific genes listed above, in at least one, and often in several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). If additional flanking sequence are useful in optimizing expression, such sequences can be cloned using the existing sequences as probes. Mammary-gland specific regulatory sequences from different organisms can be obtained by screening libraries from such organisms using known cognate nucleotide sequences, or antibodies to cognate proteins as probes.

### Signal Sequences

Useful signal sequences are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the milk-specific promoter used in the expression system of this invention. The size of the signal sequence is not critical for this invention. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin are useful in the present invention. A preferred signal sequence is the goat β-casein signal sequence.

Signal sequences from other secreted proteins, e.g., immunoglobulins, or proteins secreted by liver cells, kidney cell, or pancreatic cells can also be used.

### Insulator Sequences

The DNA constructs of the invention further comprise at least one insulator sequence. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3′ end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

### DNA Constructs

A fusion protein can be expressed from a construct which includes a promoter specific for mammary epithelial cells, e.g., a casein promoter, e.g., a goat beta casein promoter, a milk-specific signal sequence, e.g., a casein signal sequence, e.g., a β-casein signal sequence, and a DNA encoding a fusion protein.

A construct can also include a 3′ untranslated region downstream of the DNA sequence coding for the non-secreted protein. Such regions can stabilize the RNA transcript of the expression system and thus increases the yield of desired protein from the expression system. Among the 3′ untranslated regions useful in the constructs of this invention are sequences that provide a poly A signal. Such sequences may be derived, e.g., from the SV40 small t antigen, the casein 3′ untranslated region or other 3′ untranslated sequences well known in the art. Preferably, the 3′ untranslated region is derived from a milk specific protein. The length of the 3′ untranslated region is not critical but the stabilizing effect of its poly A transcript appears important in stabilizing the RNA of the expression sequence.

A construct can include a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

A construct can also include about 10%, 20%, 30%, or more of the N-terminal coding region of a gene preferentially expressed in mammary epithelial cells. For example, the N-terminal coding region can correspond to the promoter used, e.g., a goat β-casein N-terminal coding region.

Prior art methods can include making a construct and testing it for the ability to produce a product in cultured cells prior to placing the construct in a transgenic animal. Surprisingly, the inventors have found that such a protocol may not be of predictive value in determining if a normally non-secreted protein can be secreted, e.g., in the milk of a transgenic animal. Therefore, it may be desirable to test constructs directly in transgenic animals, e.g., transgenic mice, as some constructs which fail to be secreted in CHO cells are secreted into the milk of transgenic animals.

### Purification from Milk

The transgenic fusion protein can be produced in milk at relatively high concentrations and in large volumes, providing continuous high level output of normally processed peptide that is easily harvested from a renewable resource. There are several different methods known in the art for isolation of proteins from milk.

Milk proteins usually are isolated by a combination of processes. Raw milk first is fractionated to remove fats, for example, by skimming, centrifugation, sedimentation (H. E. Swaisgood, Developments in Dairy Chemistry, I: Chemistry of Milk Protein, Applied Science Publishers, NY, 1982), acid precipitation (U.S. Pat. No. 4,644,056) or enzymatic coagulation with rennin or chymotrypsin (Swaisgood, ibid.). Next, the major milk proteins may be fractionated into either a clear solution or a bulk precipitate from which the specific protein of interest may be readily purified.

U.S. Ser. No. 08/648,235 discloses a method for isolating a soluble milk component, such as a peptide, in its biologically active form from whole milk or a milk fraction by tangential flow filtration. Unlike previous isolation methods, this eliminates the need for a first fractionation of whole milk to remove fat and casein micelles, thereby simplifying the process and avoiding losses of recovery and bioactivity. This method may be used in combination with additional purification steps to further remove contaminants and purify the component of interest.

### Production of Transgenic Protein in the Eggs of a Transgenic Animal

A fusion protein can be produced in tissues, secretions, or other products, e.g., an egg, of a transgenic animal. For example, fusion proteins can be produced in the eggs of a transgenic animal, preferably a transgenic turkey, duck, goose, ostrich, guinea fowl, peacock, partridge, pheasant, pigeon, and more preferably a transgenic chicken, using methods known in the art (Sang et al., Trends Biotechnology, 12:415-20, 1994). Genes encoding proteins specifically expressed in the egg, such as yolk-protein genes and albumin-protein genes, can be modified to direct expression of fusion protein.

### Egg Specific Promoters

Useful transcriptional promoters are those promoters that are preferentially activated in the egg, including promoters that control the genes encoding egg proteins, e.g., ovalbumin, lysozyme and avidin. Promoters from the chicken ovalbumin, lysozyme or avidin genes are preferred. Egg-specific protein promoters or the promoters that are specifically activated in egg tissue can be from cDNA or genomic sequences. Preferably, the egg-specific promoters are genomic in origin.

DNA sequences of egg specific genes are known in the art (see, e.g., Burley et al., “The Avian Egg”, John Wiley and Sons, p. 472, 1989, the contents of which are incorporated herein by reference). If additional flanking sequence are useful in optimizing expression, such sequences can be cloned using the existing sequences as probes. Egg specific regulatory sequences from different organisms can be obtained by screening libraries from such organisms using known cognate nucleotide sequences, or antibodies to cognate proteins as probes.

### Transgenic Plants

A fusion protein can be expressed in a transgenic organism, e.g., a transgenic plant, e.g., a transgenic plant in which the DNA transgene is inserted into the nuclear or plastidic genome. Plant transformation is known as the art. See, in general, Methods in Enzymology Vol. 153 (“Recombinant DNA Part D”) 1987, Wu and Grossman Eds., Academic Press and European Patent Application EP 693554.

Foreign nucleic acid can be introduced into plant cells or protoplasts by several methods. For example, nucleic acid can be mechanically transferred by microinjection directly into plant cells by use of micropipettes. Foreign nucleic acid can also be transferred into a plant cell by using polyethylene glycol which forms a precipitation complex with the genetic material that is taken up by the cell (Paszkowski et al. (1984) EMBO J. 3:2712-22). Foreign nucleic acid can be introduced into a plant cell by electroporation (Fromm et al. (1985) Proc. Natl. Acad. Sci. USA 82:5824). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells with the transformed gene can be accomplished using phenotypic markers.

Cauliflower mosaic virus (CaMV) can be used as a vector for introducing foreign nucleic acid into plant cells (Hohn et al. (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule which can be propagated in bacteria. The recombinant plasmid can be further modified by introduction of the desired DNA sequence. The modified viral portion of the recombinant plasmid is then excised from the parent bacterial plasmid, and used to inoculate the plant cells or plants.

High velocity ballistic penetration by small particles can be used to introduce foreign nucleic acid into plant cells. Nucleic acid is disposed within the matrix of small beads or particles, or on the surface (Klein et al. (1987) Nature 327:70-73). Although typically only a single introduction of a new nucleic acid segment is required, this method also provides for multiple introductions.

A nucleic acid can be introduced into a plant cell by infection of a plant cell, an explant, a meristem or a seed with Agrobacterium tumefaciens transformed with the nucleic acid. Under appropriate conditions, the transformed plant cells are grown to form shoots, roots, and develop further into plants. The nucleic acids can be introduced into plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al. (1984) “Inheritance of Functional Foreign Genes in Plants,” Science 233:496-498; Fraley et al. (1983) Proc. Natl. Acad. Sci. USA 80:4803).

Plants from which protoplasts can be isolated and cultured to give whole regenerated plants can be transformed so that whole plants are recovered which contain the transferred foreign gene. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Plant regeneration from cultured protoplasts is described in Evans et al., “Protoplasts Isolation and Culture,” Handbook of Plant Cell Cultures 1:124-176 (MacMillan Publishing Co. New York 1983); M. R. Davey, “Recent Developments in the Culture and Regeneration of Plant Protoplasts,” Protoplasts (1983)-Lecture Proceedings, pp. 12-29, (Birkhauser, Basal 1983); P. J. Dale, “Protoplast Culture and Plant Regeneration of Cereals and Other Recalcitrant Crops,” Protoplasts (1983)-Lecture Proceedings, pp. 31-41, (Birkhauser, Basel 1983); and H. Binding, “Regeneration of Plants,” Plant Protoplasts, pp. 21-73, (CRC Press, Boca Raton 1985).

Regeneration from protoplasts varies from species to species of plants, but generally a suspension of transformed protoplasts containing copies of the exogenous sequence is first generated. In certain species, embryo formation can then be induced from the protoplast suspension, to the stage of ripening and germination as natural embryos. The culture media can contain various amino acids and hormones, such as auxin and cytokinins. It can also be advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In vegetatively propagated crops, the mature transgenic plants can be propagated by the taking of cuttings or by tissue culture techniques to produce multiple identical plants for trialling, such as testing for production characteristics. Selection of a desirable transgenic plant is made and new varieties are obtained thereby, and propagated vegetatively for commercial sale. In seed propagated crops, the mature transgenic plants can be self crossed to produce a homozygous inbred plant. The inbred plant produces seed containing the gene for the newly introduced foreign gene activity level. These seeds can be grown to produce plants that have the selected phenotype. The inbreds according to this invention can be used to develop new hybrids. In this method a selected inbred line is crossed with another inbred line to produce the hybrid.

Parts obtained from a transgenic plant, such as flowers, seeds, leaves, branches, fruit, and the like are covered by the invention, provided that these parts include cells which have been so transformed. Progeny and variants, and mutants of the regenerated plants are also included within the scope of this invention, provided that these parts comprise the introduced DNA sequences. Progeny and variants, and mutants of the regenerated plants are also included within the scope of this invention.

Selection of transgenic plants or plant cells can be based upon a visual assay, such as observing color changes (e.g., a white flower, variable pigment production, and uniform color pattern on flowers or irregular patterns), but can also involve biochemical assays of either enzyme activity or product quantitation. Transgenic plants or plant cells are grown into plants bearing the plant part of interest and the gene activities are monitored, such as by visual appearance (for flavonoid genes) or biochemical assays (Northern blots); Western blots; enzyme assays and flavonoid compound assays, including spectroscopy, see, Harborne et al. (Eds.), (1975) The Flavonoids, Vols: 1 and 2, [Acad. Press]). Appropriate plants are selected and further evaluated. Methods for generation of genetically engineered plants are further described in U.S. Pat. No. 5,283,184, U.S. Pat. No. 5, 482,852, and European Patent Application EP 693 554, all of which are hereby incorporated by reference.

Embodiments of the invention are further illustrated by the following examples which should not be construed as being limiting. The contents of all cited references (including literature references, issued patents, published patent applications, and co-pending patent applications) cited throughout this application are hereby expressly incorporated by reference.

### EXAMPLE 1

### Generation and Testing of An Antibody-Carboxypeptidase B Fusion

An F(ab′) 2-enzyme fusion protein was subcloned into a Goat Beta-Casein expression vector BC350. For each one of the 3 constructs: 213 (MF21q3-13, Fd-enzyme fusion gene), LC (LC3, light chain), and 141 (MF141-4, pro domain with C-terminal leucine), expression cassettes were separated from the bacterial plasmid sequences. The three transgenes were then co-microinjected in mouse zygotes. Seven transgenic mouse lines that carry all 3 subunits of the F(ab′)2-enzyme fusion protein antibody and 3 lines that only carried transgenes LC and 213 were analyzed. Milk samples were collected from founder and first generation females, and tested for ELISA and enzyme activity assays. Four of the seven lines carrying 3 transgenes express the F(ab′)2-enzyme fusion protein at levels superior to 1 mg/ml (possibly up to 4-6 mg/ml), whereas all 3 lines carrying only the LC and 213 transgenes express at levels inferior to 0.1 mg/ml.

Transgenic mice expressing a humanized antibody fragment—enzyme fusion protein (F(ab′)2-CPB) comprising a humanized anti-carcinoembryonic antigen (CEA) F(ab′)2, 806.077 fused to a modified human carboxypeptidase B enzyme were generated. These transgenic mice were generated by co-microinjection of three Goat Beta-Casein mammary gland expression constructs. One construct, 141 (MF141-4, pro domain with C-terminal leucine) expressed the pro-domain of CPB, the other 2 constructs, LC and 213 (light chain and Fd-enzyme fusion gene respectively) expressed-the antibody-CPB fusion. Expression of the CPB pro-domain in trans was shown in experiments conducted previously to be necessary for the proper folding of fusion-proteins based on mature CPB.

### Materials and Methods

Restriction enzymes were obtained from New England Biolabs, Beverly, Mass. Nylon membranes (MagnaGraph nylon transfer membranes) were obtained from Micron Sepasrations Inc (MSI, Westboro, Mass. 01581). Alpha³²P—dATP was obtained from NEN Life Science Products, Inc. Boston, Mass. Sequencing was performed by Sequegen Company, Worcester, Mass. Plasmids, 213 containing the MF21q3-13 Fd-enzyrne fusion gene, LC containing the 806.077 light chain coding region, and 141 Zeneca Pharmaceuticals. CD1 mice were obtained from Charles River Labs, Wilmington, Mass.

### Preparation of Injection Fragments

Plasmid DNA was obtained from Dr. Michael D. Edge (Zeneca Pharmaceuticals) and expression cassettes (100 μg each) were separated from the vector backbone by digesting to completion with SalI. Digests were then electrophoresed in an agarose gel, using 1× TAE (Maniatis et al., 1982) as running buffer. The region of the gel containing the DNA fragment corresponding to the expression cassette was visualized under UV light (long wave). The band containing the DNA of interest was excised, transferred to a dialysis bag; and the DNA is isolated by electro-elution in 1× TAE. This procedure was applied for each expression cassette.

Following electro-elution, DNA fragments were concentrated and cleaned-up by using the “Wizard DNA clean-up system” (Promega, Cat #A7280), following the provided protocol and eluting in 125 ml of microinjection buffer (10 mM Tris pH 7.5 EDTA 0.2 mM0. Fragment concentration was evaluated by comparative agarose gel electrophoresis. The deduced concentrations of microinjection fragments stocks were as follows: LC, 15 ng/ml; 141, 180 ng/ml, and 213, 270 ng/ml. The stocks were co-diluted in microinjection buffer just prior to pronuclear injections so that the final concentration of each fragment was 0.5 ng.ml.

### Microinjection

CD1 female mice were superovulated and fertilized ova were retrieved from the oviduct. The male pronuclei were then microinjected with DNA diluted in microinjection buffer. Microinjected embryos were either cultured overnight in CZB media or transferred immediately into the oviduct of pseudopregnant recipient CD1 female mice. Twenty to thirty 2-cell or forty to fifty one-cell embryos were transferred to each recipient female and allowed to proceed to term.

### Identification of Founder Animals

Genomic DNA was isolted from tail tissue by precipitation with sopropanol and analyzed by polymerase chain reaction (PCR) for the presence of the chicken beta-globin insulator DNA sequence. This sequence is part of the Goat Beta-Case vector (GBC 350). For the PCR reactions, approximately 250 ng of genomic DNA is diluted in 50 μl of PCR buffer (20 mM Tris pH 8.3, 50 mM KCl and 1.5 mM MgCL₂, 100 μM deoxynucleotide triphosphates, and each primer at a concentration of 600 nM) with 2.5 units of Taq polymerase and processed using the following temperature program

| | | |
|---|---|---|
| 1 cycle | 94° | 60 sec |
| 5 cycles | 94° C. | 30 sec |
| | 58° C. | 45 sec |
| | 74° C. | 45 sec |
| 30 cycles | 94° C. | 30 sec |
| | 55° C. | 30 sec |
| | 74° C. | 30 sec |

Primer Sets:

GBC 332 and GBC 386, amplicon is 206 bp

| | | |
|---|---|---|
| GBC 332: | TGTGCTCCTCTCCATGCTGG | (SEQ ID NO:_{——}) |
| GBC 386: | TGGTCTGGGGTGACACATGT | (SEQ ID NO:_{——}) |

### Southern Blot Analysis of Transgenic Founders

Genomic DNA ((24 μg total, 8 μg/lane) from each founder mouse positive for the insulator PCR was digested to completion with the restriction enzyme EcoRI. Digested DNAs were electrophoresed in triplicate and transferred to nylon membranes according to standard methods (Maniatis et al., 1982). Probes specific for each expression cassette were isolated from the VK (LC10 in pSP72, 72 bp probe), ProL (pMF141-4 in pSP72 345, bp probe), and fd-CPB (pMF213-20 in pSP72, 1861 bp probe) plasmids (provided by Michael D. Edge, Zeneca Pharmaceuticals) by cutting with SalI, Xhol, and Xhol respectively. Each probe was labeled using reagents from the Prime-It″II kit (Stratagene, LaJolla Calif. 92037) according to manufacturers' instructions, and hybridized to one set of nylon filters in 50% formamide at 42° C. following standard protocols (Maniatis et al., 1982). Washes were performed at 60° C., with 0.2× SSC, 0.1% SDS.

### Mouse Milking

Female mice were allowed to deliver their pups naturally, and were generally milked on days 7 and 9 postpartum. Mice were separated from their litters for approximately one hour prior to the milking procedure. After the one hour holding period, mice were induced to lactate using an intraperitoneal injection of 5 i. U. Oxytocin in sterile Phosphate Buffered Saline, using a 25 gaugage needle. Hormone injections were followed by a one to five minutes waiting period for the Oxytocin to take effect. A suction and collection system consisting of a 15 ml conical tube sealed with a rubber stopper with two 18 gauge needles inserted in it, the hub end of one needle being inserted into rubber tubing connected to a human breast pump, was used for milking. Mice were placed on a cage top, held only by their tail and otherwise not restricted or confined. The hub end of the other needle was placed over the mice's teats (one at a time) for the purpose of collecting the milk into individual eppendorf tube placed in the 15 ml conical tube. Eppendorf tubes were changed after each sample collection. Milking was continued until at least 150 μl of milk had been obtained. After collection, mice were returned to their litters.

### Microinjection of Mouse Embryos

The fragments were coinjected into 1708 mouse embryos, of which 945 were transferred to 31 recipient females. Of these females, 27 carried pups to term and gave birth to 172 p ups, 20 of which appeared transgenic following PCR analysis. Of the embryos injected, 1.2% appeared transgenic; of the pups born, 11.6% appeared transgenic.

### Southern Blot Analysis of Founder Mouse Lines

The 20 transgenic founders identified with the insulator PCR were analyzed further by Southern blotting hybridization to determine: A—which were positives for all three (35, 63, 73, 81, 86, 92, 120, 169) were weak mosaics. These were clearly positive using the very sensitive PCR assay, but no equivocal positive signal could be detected using Southern hybridizations. Six other founders (5, 76, 121, 128, 131, and 161 were clearly positive for at least one of the transgenes, but clearly negative or mosaic for at least one of the other transgenes. Finally, six founders (25, 67, 89, 106, 161, 166) showed hybridization signals indicating at least one copy of each transgene.

TABLE 1
Summary of Southern hybridization data from Beta-
casein - F(ab′)2-enzyme fusion protein transgenic
founders. Copy number was roughly evaluated by
comparison to signal obtained with known amount of
Eco RI digested microinjection fragment (und, is
undetectable by Southern).

| | LC transgene | 141 transgene | 213 transgene |
|---|---|---|---|
| Founder | Estimated copy # | estimated copy # | estimated copy # |
| 5 | 2 | und | 2-3 |
| 25 | 4 | 4 | 2 |
| 35 | und. | und. | und. |
| 63 | und. | und. | und. |
| 67 | 2-3 | 2-3 | 3 |
| 73 | und. | und. | <1 |
| 76 | und. | 1-2 | 1 |
| 81 | und. | und. | und. |
| 86 | und. | und. | und. |
| 89 | >10 | >10 | >10 |
| 92 | und. | und. | <1 |
| 106 | 3 | 2-3 | 1 |
| 120 | und. | und. | und. |
| 121 | <1 | 1 | 1 |
| 128 | <1 | und. | <1 |
| 131 | <1 | und. | <1 |
| 152 | 2-3 | 2-3 | 2-3 |
| 161 | und. | 1 | <1 |
| 166 | 2-3 | 3 | 3-5 |
| 169 | und. | und. | und. |

### Breeding of Mouse Lines

Following Southern blot analysis of founders 10 lines were selected for breeding: 5, 25, 67, 76, 89, 106, 121, 128, 152, and 166. Table 2 summarizes the breeding of each line; Table 3, summarizes the Southern blot analysis of PCR positive F1 offspring. From this analysis, all founders, except #121, passed their transgenic integration(s) to the next generation. Other lines (5, 25, 76, 128, see Table 2) also showed signs of germline mosaicism, with low percentage of transgene positive offspring.

Southern analysis also suggested that some of the founders may have multiple integrations for some of the transgenes. For example, 200 and 201 which are offspring of founder 166 appear to have different copy number for transgenes LC and 141, and the same copy number for transgene 213. One explanation could be that the 166 founder has at least two integration sites on different chromosomes, one containing only LC and 141 transgenes and the other containing all three transgenes. 200 would have inherited both integration sites whereas 201 may have inherited only the site with all the transgenes (other scenarios are also possible). Multiple integrations are difficult to identify by Southern blot analysis, especially when 3 different transgenes are involved. However, in large animals our use of FISH (fluorescence in situ hybridization) and karyotyping permits to sort out multiple integration situations.

In summary, 2 founders (5, 76) passed double transgene integrations (LC and 213) to their offspring, and 6 lines (25, 67, 89, 106, 152, and 166) passed all three transgenes to the next generation. Another founder, 128 was doubly transgenic for LC and 213, had a transgenic offspring (232). However this offspring was not analyzed (it was born later due to delays in breeding 128). That line was not pursued further since protein analysis of 128 milk showed no significant production of the fusion-protein.

TABLE 2
Breeding of transgenic founders. All offspring were analyzed with the
insulator PCR-assay

| | PCR positive | ID number of |
|---|---|---|
| | offspring/litter | selected F1 |
| Founder | (only females | transgenic |
| (sex) | were analyzed) | females |
| 5 (F) | 2/10 | 217, 219 |
| 25 (F) | 1/7 | 204 |
| 67(M) | 1/3 | 177 |
| 76(F) | 1/6 | 212 |
| 89(F) | 2/5 | 178, 179 |
| 106(M) | 1/5 | 186 |
| 121(M) | 0/5 | None |
| 128(F) | 1/8 | 232 |
| 152(M) | 2/4 | 194, 195 |
| 166(F) | 2/6 | 200, 201 |

TABLE 3
Summary of Southern hybridization data from Beta-
casein - F(ab′)2-enzyme fusion protein transgenic F1.
Copy number was roughly evaluated by comparison to signal
obtained with know amount of EcoRI digested microinjection
fragment (und. Is undetectable by Southern).

| Founder | Transgenic | Lc Transgene | 141 Transgene | 213 Transgene |
|---|---|---|---|---|
| Parent | F1 | Copy # | Copy # | Copy # |
| 5 | 217, 219 | 2 | 0 | 2 |
| 25 | 204 | 3 | 3 | 33-4 |
| 67 | 177 | 1 | 1-2? | 1-2? |
| 76 | 212 | 2 | 0 | 2 |
| 89 | 178, 179 | >10 | >10 | >10 |
| 106 | 186 | 3 | 3-4 | 2-3 |
| 152 | 194, 195 | 4-5 | 4 | 4-5 |
| 166 | 200, 201 | 1 (200 | 1 (200 | 4-5 |
| | | 3-4 (201) | 4 (200) | (Both 200 and |
| | | | | 201) |

### Analysis of Transgenic Mouse Milk Samples

Mouse milk samples were collected from founder females as well as from F1 transgenic females. It was decided not to dilute the milk with PBS, to avoid possible interference with the enzymatic assays. Samples were frozen at −20° C. until testing. Assays are summarized below as Table 4.

TABLE 4
Summary of ELISA and activity assays performed on the
milk of mice expressing a humanized antibody fragment -
enzyme fusion protein (Fab′)2-CPB). (NA, not
applicable)

| | | | Enzyme |
|---|---|---|---|
| Founder (sex, | | ELISA levels | assay |
| Transgenes) | F1 (transgenes) | (mg/ml) | (mg/ml) |
| 5 (F, LC-213) | 217, 219 (LC-213) | 0.092 | 0.025 |
| | | low | low |
| 25 (F, LC-213-141) | 204 (LC-213-141) | 1.5* | 1.2* |
| | | 1.5-2 | 1.5-2 |
| 67 (M, LC-213-141) | 177 (LC-213-141) | NA | NA |
| | | Negative | negative |
| 76 (F, LC-213) | 212 (LC-213) | negative | negative |
| | | negative | negative |
| 89 (F, LC-213-141) | 178, 179 (LC-213- | 1.5-2 | 1.5-2 |
| | 141) | 1.5-2 | 1.5-2 |
| 106 (M, LC-213- | 186 (LC-213-141) | NA | NA |
| 141) | | 4-6 | 4-6 |
| 128 (F, LC-213) | | low | low |
| 152 (M, LC-213- | 195 (LC-213-141) | NA | NA |
| 141) | | 4-6 | 4-6 |
| 166 (F, LC-213-141) | 200, 201 (LC-213- | negative | negative |
| | 141) | negative | negative |

*Assays performed on milk collected on the second lactation of the 25 consistently gave higher values

Constructs linking the Goat Beta Casein regulatory sequences to coding region of the light and heavy chains of humanized anti-CEA F(ab′)2, 806.077 fused to a modified human carboxypeptidase B enzyme, and to the coding region of the pro-domain of CPB (with C-terminal leucine) were generated. Transgenic mouse lines were generated with and without the transgene expressing the CPB pro-domain. It was demonstrated that mice transgenic for all 3 constructs are capable of producing the (Fab′)2-CPB fusion at high levels (up to 4-6 mg/ml) in the milk of transgenic mice (4/6 triple transgenic lines expressed at levels superior to 1 mg/ml), with expected enzymatic activity. However, the absence of CPB pro-domain expression seems to correlate with low level secretion of the active fusion protein. However this result has to be considered with caution since only 3 double transgenic lines were analyzed (only 2 both founder and F1).

In summary, variants of human pancreatic carboxypeptidase B (HCPB), with specificity for hydrolysis of C-terminal glutamic acid and aspartic acid, were prepared by site-directed mutagenesis of the human gene and expressed in the periplasm of Escherichia coli. By changing residues in the lining of the S1′ pocket of the enzyme, it was possible to reverse the substrate specificity to give variants able to hydrolyse prior to C-terminal acidic amino acid residues instead of the normal C-terminal basic residues. This was achieved by mutating Asp253 at the base of the S1′ specificity pocket, which normally interacts with the basic side-chain of the substrate, to either Lys or Arg. The resulting enzymes had the desired reversed polarity and enzyme activity was improved significantly with further mutations at residue 251. The [G251T,D253K]HCPB double mutant was 100 times more active against hippuryl-L-glutamic acid (hipp-Glu) as substrate than was the single mutant. [D253K]JCPB, Triple mutants, containing additional changes at Ala248, had improved activity against hipp-Glu subtrate when position 251 was Asn. These reversed polarity mutants of a human enzyme have the potential to be used in antibody-directed enzyme prodrug therapy of cancer.

### EXAMPLE 2

### Generation and Testing of Anti-Transferrin Receptor Antibody/Angiogenin Fusion Constructs

This Examples shows expression of anti-transferrin receptor antibody/angiogenin fusion proteins in the mammary gland of transgenic mice. A chimeric mouse/human antibody directed against the human transferrin receptor (E6) was fused as its CH2 domain to the gene for a human angiogenic ribonuclease, angiogenin (Ang). It was expressed in the mammary gland of mice and secreted into mouse milk. Expression levels in milk were approximately 0.8 g/L. The chimeric protein retained antibody binding activity and protein synthesis inhibitory activity equivalent to that of free Ang. It was specifically cytotoxic to human tumor cells in vitro.

### Materials and Methods

### Transgenic Mice

Transgenic mice were generated following standard published procedures (Roberts et al., 1992; DiTullio et al., 1992; Gutierrez et al., 1996). Founder mice were bred to produce lactating females and the milk collected and diluted with an equal volume of phosphate buffered saline as previously described. Milk was stored at −70 C.

### Fractionation of Milk

Milk containing E6 IG antibody was applied to a Protein A Sepharose column and eluted with 0.1M glycine. pH 3.0 into tubes containing IM Tris based to adjust pH to neutrality. Milk containing the fusion protein (CH2Ang) was made 0.2 M EDTA and incubated on ice for 20 min before centrifugation for 10 min at 4 C in an eppifuge. The skim milk was removed carefully from the fat layer and centrifuged again before purification by size exclusion high performance liquid chromatography on a TSK 3000 column (Toso Haas Corp., Pa.) equilibrated and eluted with 0.1 M phosphate buffer, pH 7.4. The flow rate was 0.5 ml/min and 1 min fractions were collected. the majority of material reacting with an antibody against angiogenin eluted in the void volume of the column. This material was pooled and arginine powder was added to a final concentration of 1 M. After an overnight incubation at 4 C, the sample was re-chromatographed on the TSK 3000 column as described above. CH2Ang containing milk required a second treatment with 1 M arginine and re-chromatography on the sizing column.

### Protein Determination

Protein was determined using the following extinction coefficients: E6 IgG antibody, E1%/280nm=14.0; CH2Ang, E1%/280nm=10.0.

### Protein Synthesis Assay

Cells were plated at 2500 cells per well in 96-well microtiter plates in Dulbecco's minimum essential medium supplemented with 10% fetal bovine serum. Additions were made in a total volume of 10 μL, and the plates were incubated at 37 C for 3 days before 0.1 mCi of [¹⁴C]-leucine was added for 2-4 h. Cells were harvested onto glass fiber filters using a PHD cell harvester, washed with water, dried with ethanol and counted. The results are expressed a percent of [¹⁴C]-leucine incorporation in mock-treated wells.

### EXAMPLE 3

### Expression of an Antihuman Transferrin Receptor Antibody and Antibody-Angiogenin Fusion Protein in the Milk of Transgenic Mice

The DNA constructs used to produce the transgenic mice are illustrated in FIG. 1 and FIG. 2A. The chimeric antitransferin receptor antibody used in the studies described was originally fused to human tumor necrosis factor (Hoogenboom et al., 1991) and then to human ribonuclease, angiogenin (Ang, Rybak, et al., 1992). The Ang gene was fused behind the first three amino acid residues of 5′ region of the CH2 domain of the antibody, thus leaving the hinge region unaffected and dimeriaation of the heavy chain possible. The goal was to create humanized immunotoxin-like proteins that might elicit less immunogenic side effects when administered to patients. The in vivo mammalian cell expression systems yielded very little material functional studies, especially when the antibody was fused to the human Rnase, angiogenin (Ang) Ang is a member of the RNase A superfamily. All members of this superfamily are small (12-14 kDa). basic ribonucleolyutic enzymes found in the pancreas as well as other organs. Fluid and tissues of mammals and amphibians. Though these RNase can leave RNA physiological actions e.g. eliciting angiogenesis, host defense actions and antiviral effects have been described for various RNase members. Because RNases might be part of a natural defense system they have been used to create chemical conjugates and recombinant fusion protein with a variety of antibodies. Since those studies indicate that RNase based therapeutic may have potential for the treatment of cancer and AIDS, the original RNase work with the chimeric antibody against the human transferrin receptor was re-explored using newly developed technology for the production of complex proteins in the milk transgenic animals. The molecular details of the genetic constructs used in these studies are shown above. The Roman numerals correspond to those shown in FIG. 2 panel A and expand on the DNAs cloned between exons 2 and 7 of the goat β-casein gene.

DNA encoding the entire heavy chain of the E6 antibody, a chimeric antibody against the human transferrin receptor (Hoogenboom et al., 1990) was used between exons 2 and 7 of a modified goat B-casein gene (FIG. 2A, I) that is expressed at high levels in the milk of lactating transgenic mice (Roberts et al., 1992). A second transgene encoding an antibody-enzyme fusion was prepared by linking the gene for the human RNase, angiogenin (Ang) to the CH2 domain of the antibody (FIG. 1 and FIG. 2A, II). Those genes as well as the gene encoding the light chain of the same antibody (FIG. 2A, III) were all cloned separately, and the appropriate pairs (heavy (H) and light (L) chains; CH2Ang and L chain) were purified free of procaryotic DNA and co-injected into mouse embryos that were reimplanted using standard methods (Roberts et al., 1992). Transgenic mice were identified by PCR and southern blot analysis of DNA obtained from tails of the resulting progeny.

Founder mice were bred to produce lactating transgenic females. Milk was collected, diluted with PBS and analyzed for the presence of the antibody chains and Ang. Polyclonal antibodies raised against human Ang only reacted with a band of the expected M (43 kDa; antibody heavy chain, 29 kDA; Ang, 14 kDA) in the fusion protein (FIG. 2B, left panel). However, anti-IgG antisera strongly reacted with both the H and L chains of the chimeric E6 antibody (FIG. 2B, right panel). Whereas the L chain of the antibody fusion protein was clearly observed with the anti-IgG antisera, the truncated H chain of CH2Ang was barely detectable suggesting that the fusion of angiogenin to the CH2 domain hindered binding of the antisera to the H chain.

The chimeric IgG antibody was purified by chromatography on Protein A Sepharose. As shown in FIG. 2C, lanes 1 and 2, Western analysis of the final purified product by gel electrophoresis under reducing conditions showed the presence of light (28 kDa) and heavy chain proteins (approximately 55 kDa). Western analysis under non-reducing conditions (FIG. 2C, lane 3) demonstrated that the transgenic antibody existed as a mixture of IgG and Fab forms (168 and 84 kDa, respectively). A small amount of free heavy chain (55 kDa) was also seen.

Milk containing the CH2Ang fusion protein was similarly collected and diluted with PBS. Protein A Sepharose failed to bind the angiogenin fusion protein. Analogous results were obtained when the same CH2 antibody fragment previously was fused to TNF and it was postulated that this was due to the deletion of the Protein A binding site believed to be near the CH2-CH3 junction (Hoogenboom et al., 1991). The nature of the transgenic antibody-Ang fusion protein was determined by Western blotting. After reduction of the interchain disulfide bonds, the H chain Ang fusion (43 kDA) and light chain (28 kDA) were dissociated (FIG. 2C, lane 2). Western analysis with an anti-IgG antibody under non-reducing conditions (FIG. 2C, lane 5) demonstrated that the transgenic antibody-enzyme fusion protein existed as a mixture of F(ab)₂ and Fab forms (142 and 71 kDa, respectively). Identical results were obtained when the analysis was performed with anti-Ang antisera (not shown). Taken together the latter results demonstrate that the light chain is associated with the heavy chain-Ang fusion.

### EXAMPLE 4

### Biological Characterization of Antibody-angiogenin Fusion Protein

Angiogenin is a potent inhibitor of the translational capacity of the rabbiteticulocyte lysate by a mechanism that depends upon its ribonucleoytic activity (St. Clair et al., 1987). FIG. 2 shows that the addition of Ang or CH2Ang to the lysate caused the inhibition of protein synthesis as measured by the incorporation of [³⁵S]methionine into acid-precipitable protein. The IC₅₀S(40 nM) of unfused Ang or CH2Ang were indistinguishable in this assay indicating that the conformation of the active site residues was not affected by fusing Ang in this orientation (NH₂-terminus) to the CH2 antibody domain.

The antibody potion of the fusion protein was characterized by competition binding experiments (Table 5). Binding of milk-derived E6 antibody (IgG) to the human transferrin receptor was tested and compared to that of the same antibody originally purified from hybridoma cells (Heyligen et al., 1985). The ability of both antibodies to displace the [¹²⁵I]labeled parental antibody was identical (50% displacement by either antibody was 0.8 nM). the CH2Ang fusion protein was 175 fold less active than the E6 intact antibody (140 nM CH2Ang versus 0.8 nM E6).

The cytotoxic effects of the Ang fusion protein on human tumor cells was assessed by measuring [¹⁴C]leucine incorporation into newly synthesized proteins. Typical dose response curves are depicted in FIG. 3. CH2Ang inhibited the protein synthesis of SFS39 human glioma cells and MDA-MB-231^{mdrl} breast cancer cells with IC₅₀^{S} of 15 and 45 nM, respectively. Cytoxicity on other human tumor cell lines is compared in Table II. The IC₅₀^{S} ranged from 15 to 70 nM. Cytotoxicity was specific to the fusion protein since no activity was observed on an antigen negative cell line (mouse NIH3T3 cells, data not shown) and a five fold molar excess of the unfused chimeric antibody reversed cytotoxicity by approximately 50%. Whereas CH2Ang inhibited protein synthesis to 99% of mock treated cells, protein synthesis was decreased to 45% of mock treated cells in the presence of a 5 fold molar excess of antibody. Since neither the unfused antibody (Rybak et al., 1992) or free Ang (Newton et al., 1996) are cytotoxic, the two domains in the fusion proteins must be covalently joined to elicit cytotoxic, the two domains in the fusion proteins must be covalently joined to elicit cytotoxicity.

Angiogenin was isolated from tumor cell conditioned medium by following angiogenic activity in the chicken embryo chorioallantoic membrane and rabbit corneal assays (Fett et al., 1985). Its homology to ribonuclease and distinctive nucleolytic activity (Shapiro et al., 1986) coupled to its angiogenic activity yield unique biological properties that may promote enhanced tumor cell killing when Ang is targeted to tumor cells with cell specific targeting agents. Angiogenic activity is maintained when Ang is expressed as a fusion protein (Newton et al., 1996). Angiogenin also binds a cell surface proteoglycan on human colon carcinoma cells (Soncin et al., 1994). Accordingly, localization to tumor sites by the antibody could be increased by the tumor cell binding properties of Ang while increased angiogenesis could conceivable aid tumor penetration by increasing tumor vascularization (Newton et al., 1996). Moreover antagonists of Ang prevent tumor growth (Piccoli et al., 1998; Olson et al., 1995). Thus Ang activities are pleiotropic; their manifestation is governed by the cellular milieu to which Ang is exposed e.g., targeting the cytosolic protein synthesis machinery causes cytoxicity (St. Clair et al., 1987; Rybak et al., 1991) while endocytosis and translocation of Ang to the nucleus in endothelial cells has been reported to elicit angiogenesis (Moroianu and Riordan, 1994). These biological properties of Ang afford unique opportunities to design both cytostatic (antiangiogenic) and cytotoxic (antitumor cell) therapeutic strategies by antagonizing or specifically targeting this protein, respectively.

The realization of human enzyme-based multi-domain targeted therapeutics for cancer (Rybak et al., 1991; Rybak et al., 1992; Newton et al., 1992; Newton et al., 1994; Newton et al., 1996; Jinno et al., 1996; Zewe et al., 1997; Deonarain & Epenetos, 1998) and cardiovascular disease (Haber, 1994; Collen, 1997) depends on developing expression systems capable of producing these reagents for preclinical characterization and eventual clinical use. Expression of a two chain antibody Ang fusion protein in the milk of transgenic mice was accomplished and presented in this study. It was not obvious that Ang could be successfully expressed as a fusion protein in transgenic mice because a similar fusion protein was expressed only at very low levels from cultured myeloma cells presumably due to retrograde transport during secretion leading to the selection of low producers (Rybak et al., 1992). Remarkably, in the natural environment of the mammary gland the efficiency of expression was increased 160,000 times over the cell culture system (0.8 g/L vs. 5 μg/L in milk and myeloma cells, respectively). Thus, it was possible to purify sufficient amounts of the Ang fusion protein for biological characterization. One of the consequences of this work is that the importance of the orientation of Ang in a fusion protein is demonstrated for the first time. In single chain antibody Ang fusion proteins Ang was fused at the C-terminus to the N-terminus of the antibody (Newton et al., 1996). Subsequently, it became known that the last three amino acid residues of the C-terminal region of Ang contribute an active center subsite (Russo et al., 1996). Whereas Ang in the CH2 fusion protein and free Ang were equipotent in the rabbit reticulocyte lysate assay, Ang in a single chain fusion protein was two fold less effective than Free Ang to inhibit protein synthesis in the lysate assay (Newton et al., 1996).

This is the first demonstration, in general, that antibody-enzyme fusion proteins can be expressed at high levels in the mammary gland. In particular, the demonstration that antibody-Ang fusions can be expressed in the mammary gland has implications of the development of transgenic mouse models for breast cancer. Promoters from other milk specific genes have been used to cause the expression of transgenes during lactation imitating the onset of neoplasias (Amundadottier et al., 1996). Since the results of the present study show that a milk specific promoter can induce expression of an active immunotoxin, double transgenic strains could be developed to test whether the expression of an Ang fusion protein targeted against the engineered neoplasia could prevent or alter the progression of the disease. These results are especially relevant to Ang since murine counterparts are available (Bond et al., 1993).

In summary, these results demonstrate for the first time that complex heterologous fusion proteins can be expressed in the mammary gland of mice in larger amounts and with superior biological properties than mammalian cell culture (Rybak et al., 1992) and E. coli expression systems (Newton et al., 1996). The results impact both the possibility of producing these fusion proteins as therapeutics as well as the possibility of creating new animal models for breast cancer.

The following abbreviations are used herein Ang. human angiogenin; E6, anti-transferrin receptor IgG monolonal antibody; RNase, ribonuclease; H chain heavy chain; L chain, light chain: CH2Ang, angiogenin fused to the CH2 domain of the E6 heavy chain; IC_{50′} the concentration of fusion protein which inhibits protein synthesis by 50%.

TABLE 5
Binding of E6 and Ang fusion proteins to human transferrin receptor

| | |
|---|---|
| Binding | Bold Difference |

| | | |
|---|---|---|
| Construct | Source | EC₅₀(nM) |

| | | | |
|---|---|---|---|
| E6 | hybridoma | 0.8 | 1 |
| E6 | milk | 0.8 | 1 |
| CH2aNG | MILK | 140 | 175 |

TABLE II
Cytoxicity of Ch2Ang

| | CH2Ang |
|---|---|
| Cell Line | Ic₅₀(nM) |
| As539 | 15 |
| HS578T | 70 |
| MDA0MB-23 [mdr] | 45 |
| MALME | 40 |
| ACHN | 30 |

### EXAMPLE 7

### Generation and Characterization of Transgenic Goats

The sections outlined below briefly describe the major steps in the production of transgenic goats.

### Goat Species and Breeds

Swiss-origin goats, e.g., the Alpine, Saanen, and Toggenburg breeds, are preferred in the production of transgenic goats.

### Goat Superovulation

The timing of estrus in the donors is synchronized on Day 0 by 6 mg subcutaneous norgestomet ear implants (Syncromate-B, CEVA Laboratories, Inc., Overland Park, Kans.). Prostaglandin is administered after the first seven to nine days to shut down the endogenous synthesis of progesterone. Starting on Day 13 after insertion of the implant, a total of 18 mg of follicle-stimulating hormone (FSH—Schering Corp., Kenilworth, N.J.) is given intramuscularly over three days in twice-daily injections. The implant is removed on Day 14. Twenty-four hours following implant removal the donor animals are mated several times to fertile males over a two-day period (Selgrath, et al., Theriogenology, 1990. pp. 1195-1205).

### Embryo Collection

Surgery for embryo collection occurs on the second day following breeding (or 72 hours following implant removal). Superovulated does are removed from food and water 36 hours prior to surgery. Does are administered 0.8 mg/kg Diazepam (Valium®) IV, followed immediately by 5.0 mg/kg Ketamine (Keteset), IV. Halothane (2.5%) is administered during surgery in 2 L/min oxygen via an endotracheal tube. The reproductive tract is exteriorized through a midline laparotomy incision. Corpora lutea, unruptured follicles greater than 6 mm in diameter, and ovarian cysts are counted to evaluate superovulation results and to predict the number of embryos that should be collected by oviductal flushing. A cannula is placed in the ostium of the oviduct and held in place with a single temporary ligature of 3.0 Prolene. A 20 gauge needle is placed in the uterus approximately 0.5 cm from the uterotubal junction. Ten to twenty ml of sterile phosphate buffered saline (PBS) is flushed through the cannulated oviduct and collected in a Petri dish. This procedure is repeated on the opposite side and then the reproductive tract is replaced in the abdomen. Before closure, 10-20 ml of a sterile saline glycerol solution is poured into the abdominal cavity to prevent adhesions. The linea alba is closed with simple interrupted sutures of 2.0 Polydioxanone or Supramid and the skin closed with sterile wound clips.

Fertilized goat eggs are collected from the PBS oviductal flushings on a stereomicroscope, and are then washed in Ham's F12 medium (Sigma, St. Louis, Mo.) containing 10% fetal bovine serum (FBS) purchased from Sigma. In cases where the pronuclei are visible, the embryos is immediately microinjected. If pronuclei are not visible, the embryos can be placed in Ham's F12 containing 10% FBS for short term culture at 37° C. in a humidified gas chamber containing 5% CO2 in air until the pronuclei become visible (Selgrath, et al., Theriogenology, 1990. pp. 1195-1205).

### Microinjection Procedure

One-cell goat embryos are placed in a microdrop of medium under oil on a glass depression slide. Fertilized eggs having two visible pronuclei are immobilized on a flame-polished holding micropipet on a Zeiss upright microscope with a fixed stage using Normarski optics. A pronucleus is microinjected with the DNA construct of interest, e.g., a BC355 vector containing the human erythropoietin analog-human serum albumin (immunoglobulin-enzyme-hSA) fusion protein gene operably linked to the regulatory elements of the goat beta-casein gene, in injection buffer (Tris-EDTA) using a fine glass microneedle (Selgrath, et al., Theriogenology, 1990; pp. 1195-1205).

### Embryo Development

After microinjection, the surviving embryos are placed in a culture of Ham's F12 containing 10% FBS and then incubated in a humidified gas chamber containing 5% CO2 in air at 37° C. until the recipient animals are prepared for embryo transfer (Selgrath, et al., Theriogenology, 1990. p. 1195-1205).

### Preparation of Recipients

Estrus synchronization in recipient animals is induced by 6 mg norgestomet ear implants (Syncromate-B). On Day 13 after insertion of the implant, the animals are given a single non-superovulatory injection (400 I.U.) of pregnant mares serum gonadotropin (PMSG) obtained from Sigma. Recipient females are mated to vasectomized males to ensure estrus synchrony (Selgrath, et al., Theriogenology, 1990. pp. 1195-1205).

### Embryo Transfer

All embryos from one donor female are kept together and transferred to a single recipient when possible. The surgical procedure is identical to that outlined for embryo collection outlined above, except that the oviduct is not cannulated, and the embryos are transferred in a minimal volume of Ham's F12 containing 10% FBS into the oviductal lumen via the fimbria using a glass micropipet. Animals having more than six to eight ovulation points on the ovary are deemed unsuitable as recipients. Incision closure and post-operative care are the same as for donor animals (see, e.g., Selgrath, et al., Theriogenology, 1990. pp. 1195-1205).

### Monitoring of Pregnancy and Parturition

Pregnancy is determined by ultrasonography 45 days after the first day of standing estrus. At Day 110 a second ultrasound exam is conducted to confirm pregnancy and assess fetal stress. At Day 130 the pregnant recipient doe is vaccinated with tetanus toxoid and Clostridium C&D. Selenium and vitamin E (Bo-Se) are given IM and Ivermectin was given SC. The does are moved to a clean stall on Day 145 and allowed to acclimatize to this environment prior to inducing labor on about Day 147. Parturition is induced at Day 147 with 40 mg of PGF2a (Lutalyse®, Upjohn Company, Kalamazoo Mich.). This injection is given IM in two doses, one 20 mg dose followed by a 20 mg dose four hours later. The doe is under periodic observation during the day and evening following the first injection of Lutalyse® on Day 147. Observations are increased to every 30 minutes beginning on the morning of the second day. Parturition occurred between 30 and 40 hours after the first injection. Following delivery the doe is milked to collect the colostrum and passage of the placenta is confirmed.

### Verification of the Transgenic Nature of F₀ Animals

To screen for transgenic F₀ animals, genomic DNA is isolated from two different cell lines to avoid missing any mosaic transgenics. A mosaic animal is defmed as any goat that does not have at least one copy of the transgene in every cell. Therefore, an ear tissue sample (mesoderm) and blood sample are taken from a two day old F₀ animal for the isolation of genomic DNA (Lacy, et al., A Laboratory Manual, 1986, Cold Springs Harbor, N.Y.; and Herrmann and Frischauf, Methods Enzymology, 1987. 152: pp. 180-183). The DNA samples are analyzed by the polymerase chain reaction (Gould, et al., Proc. Natl. Acad. Sci, 1989. 86: pp. 1934-1938) using primers specific for human immunoglobulin-enzyme-hSA fusion protein gene and by Southern blot analysis (Thomas, Proc Natl. Acad. Sci., 1980. 77:5201-5205) using a random primed IMMUNOGLOBULIN-ENZYME or hSA cDNA probe (Feinberg and Vogelstein, Anal. Bioc., 1983. 132: pp. 6-13). Assay sensitivity is estimated to be the detection of one copy of the transgene in 10% of the somatic cells.

### Generation and Selection of Production Herd

The procedures described above can be used for production of transgenic founder (F₀) goats, as well as other transgenic goats. The transgenic F₀ founder goats, for exampre, are bred to produce milk, if female, or to produce a transgenic female offspring if it is a male founder. This transgenic founder male, can be bred to non-transgenic females, to produce transgenic female offspring.

### Transmission of Transgene and Pertinent Characteristics

Transmission of the transgene of interest, in the goat line is analyzed in ear tissue and blood by PCR and Southern blot analysis. For example, Southern blot analysis of the founder male and the three transgenic offspring shows no rearrangement or change in the copy number between generations. The Southern blots-are probed with immunoglobulin-enzyme fusion protein cDNA probe. The blots are analyzed on a Betascope 603 and copy number determined by comparison of the transgene to the goat beta casein endogenous gene.

### Evaluation of Expression Levels

The expression level of the transgenic protein, in the milk of transgenic animals, is determined using enzymatic assays or Western blots.

### Additional List of References

Maniatis, T., Fritsch, E. F., and Sambrook, J. 1983. Molecular Cloning, A Laboratory Manual. Cold Spring Harbor, N.Y.: Cold Spring Harbor Laboratory

Amudadottir, L. T. Merlino., and Dickson, R. B. (1996) Transgenic mouse models of breast cancer, Breast Can. Res. and Treatment 39, 119.

Bond, M. D., Strydom, D. J. and Vallee, B. L. (1993) Characterization and sequencing of rabbit pig and mouse angiogenins; discernment of functionally important residues and regions. Biochems Biophys, Acta 1162.177.

Bosslet, K., Czech J., Lorenz P., Sedlacek, H. H., Schuermann, M., and Seemann. G. (1992) Molecular and functional characterization of a fusion protein suited for timer specific prodrug activation. Br.J. Cancer 65,234.

Castilla, J., Pintado, B. Sola, I., Sanchez-Morgado, J. M. and Enjuanes, L. (1998) Engineering passive immunity in transgenic mice secreting virus-neutralizing antibodies in milk. Nature Biotechnology 16, 349.

Clark A. (1997) Gene expression in the mammary glands of transgenic animals. Biochem. Soc. Symp. 63, 133.

Collen, D. (1997) Thrombolytic Therapy. Thrombosis and Haemostasis 78.742.

Colman, A. (1996) Production of proteins of proteins in the milk of transgenic livestocks; problems, solutions and success. Am. J. clin. Nutr. 63, 639S.

Deonarain, M. P. and Epenetos, A. A. (1998) Design, characterization and antitumor cytoxicity of a panel or recombinant, mammalian ribonuclease-based immunotoxins Br. J. Cancer 77,537.

DiTullio, P. Cheng S. H. Marshall, J. Gregory, R. J. Ebert, K. M. Meade H. M. and Smith, A. E. (1992) Production of cystic fibrosis transmembrane conductance regulator in the milk of transgenic mice. Bio/Technology 10,74.

Fett, J. W., Strydom, D. J. Lobb, R. R., Alderman, E. M. Bethune, J. L. Riodan, J. F. and Vallee, B. L. (1985) Isolation and characterization of angiogenin an angiogenic protein form human carcinoma cells. Biochemistry 24,5480.

Goshom, S. C. Svensson, H. P. Kerr, D. E. Somerville, J. E. Senter, P. D. and Fell, H. P. (1993) Genetic construction, expression and characterization of a single chain anti-carcinoma antibody fused to B-lactamase, cancer Res. 53,2123.

Gutierrez., A., Meade, H. M., DiTuillio, P. Pollock, D., Harvey, M. Jimenez-Flores, R. Anderson, G. Murray, J., and Medrano, J. (1996) Expression of a bovine kappa CN cDNA in the mammary gland of transgenic mice utilizing a genomic milk protein gene as an expression cassette, Transgenic Res.5, 271.

Haber, E. (1994) Antibody-plasminogen activator conjugates and recombinant proteins. methods in Mol.Genet,5,111.

Heyligen, H., Thijs, C., Weber, W., Bosmans, E., and Raus, J. (1985) Monoclonal antibodies detecting human T cell activation antigens; development of monoclonal antibodies and expression of antigens on activated T cells and leukemic cells. Fed. Proc, 44,787.

Hoogenboom, H. R. raus, J. C. M., and Volckaert, G. (1990) Cloning and expression of a chimeric antibody directed against the human transferrin receptor. J. Immunol. 144,3211.

Hoogenboom, H. E. Volckaert, C., and Raus, J. C. M. (1991) Construction and expression of antibody-tumor necrosis factor fusion proteins. Mol. immunol. 28, 1027. Jirno, H., Ueda, M., Ozawa, S., Kikuchi, K., Ikeda, T., Enomoto, K, and Kitajima, M. (1996) Epidermal growth factor receptor dependent cytotoxic effect by an effect by an EGF-ribonuclease conjugate on human cancer cell lines: a trial for less immongenic chimeric toxin. Can. Cheomoter. Pharmacol 38,303.

Limonta, J., Pedraza, A., Rodriquez, A., Grehre, F. M., Barral, A. M., Castro, F. O. Lleonart, R., Gracia, C. A. Gavilondo, J. V., and DelaFuente, J. (1995) Production of active anto-CD6 mouse/human chimeric antibodies in the milk transgenic mice. Immontechnology 1.107.

Maga, E., and Murray J. (1995) Mammary gland expression of transgenes and the potential for altering the properties of milk. Bio/Technology 13,1452.

Moroninau, J., and Riordan, J. F. (1994) Nuclear translocation of angiogenin in proliferating endothelial cells is essential to its angiogenic activity. Proc. Natl. Acad, Sci U.S.A. 91, 1677.

Newton, D. K. Illercil, O. Laske, D. W., Oldfield, E., Rybak, S. M. and Youle, R. J. (1992) Cytoxic ribonuclease chimeras: Targeted tumoricidal activity in vivo and in vivo. J. Biol, Chem. 267,19572.

Newton, D. L. Nicholls, P. J., Rybak, S. M. and Youle, R. J. (1994) Express on and characterization of recombinant human eosinophil-derived neurotoxin and eosinophil-derived neurotoxin-anti=transferrin receptor sFv. J. Biol. Chem 269, 26739.

Newton, D. L., Xue, Y., Olson, K. A., Fett, J. W., and Rybak, S. M. (1996) Angiogenin single chain imminofusions; Influence of peptide linkers and spacer between fusion proteins domains Biochemistry 35,545.

Olson, K. A., Fett, J. W. Grench, T. C., Key M. E., and Vallee, B. L. (1995) Angiogenin antagonists prevent tumor growth in vivo. Prox. Natl. Acad. Sci. U.S.A. 92, 442.

Pastan, I. (1997) Targeted therapy of cancer with recombinant immunotoxins. Biochim Biophys Acta 1333, C1.

Piccoli, R., Olson, K. A. Vallee, B. L., and Fett, J. W. (1998) Chimeric anti-angiogenin antibody cAB 26-2F inhibits the formation of human breast cancer xenografts in a hymic mice. Proc. Natl. Acad, Sci, U.S.A. 95, 4579.

Roberts, B., DiTullio, P. Vitale, J., Hehir, K., and Gordon, K. (1992) Cloning of the goat β casein-encoding gene and expression in transgenic mice Gene 121,255.

Rodriguez, M. L. Presta, L. G., Kotts, C. E., Wirth, C. Mordenti, J., Isaka, G. Wong, W. L. T., Nuijens, A. Blackburn, B., and Carter, P. (1995) Development of a humanized disulfide stabilized anto-p185 HER2 Rv-β Lactamase fusion protein for activation of a cephalosporin doxorubiein prodrug. Cancer Res. 55, 63.

Russo, N., Nobile, V., DiDonato, A., Riordan, J. F. and Valee, B. L. (1996) The C-terminal 93,3243.

Rybak, S. M., Hoogenboom, H. R. meade, H. M., Raus, j. C. Schwartz, D., and Youle, R. J. (1992) humanization of immunotoxins. Proc. Natl. Acad. Scu USA 89, 3165.

Rybak, S. M., newton, D. L. and Xue, y. (1995) RNase immunofusions for cancer therapy. Tumor Targeting 1,141.

Rybak, S. M., Saxena, S. K., Ackerman, E. J. youle, R. J. (1991) Cytotoxic potential of ribonuclease and ribonuclease hybrid proteins. J. Biol. Chem 266, 21202.

Schein, C. H, (1997) From housekeeper to microsurgeon: The diagnostic and therapeutic potential of ribonuclease. Nature Biochemistry 15, 529.

Shapiro, R. Riordan, J. F. and Vallee, B. L. (1986) Characteristic ribonuclolyt c activity of human angiogenin. Biochemistry 25, 3527.

Siemens, N. O. Kerr, D/E/. Arnold, S. Stebbins, M. R. Vrudhula, V. M., Hellstrom, J. Hellstrom, K. E. and Senter, P. D. (1997) Construction, expression and activities of L49-sFv-β-Lactamse, a single-chain antibody fusion protein for anticancer prodrug activation. Bioconjugate Chem., 8, 510.

Sola, I., Castilla, J. Pintado, B., Sanchez-Morgado, J. M. Whitelaw, B. A. Clark, A. J., and Enjuanes, L. (1998) Transgenic mice secreting conornvirus neutralizing antibodies into the mile.J.Virol, 72,3762.

Soncin, F. Sharpiro, R., and Fett, J. W. (19940 A cell-surface proteoglycan mediates human adenocarcinoma HT-29 cell adhesion to human angiogenin. J. Biol. Chem, 269,8999.

St. Clair, D. K., Rybak, S. M. Riordan, J. F., and Vallee, B. L. (1987) Angiogenin abolishes cell-free protein synthesis by specific ribonuclolytic inactivation of biosomes. Proc. Natl. Acad. Sci. USA 84, 8330.

Strydom D. J. Fett, J. W. Lobb, R. R. Alderan, E. M. Bethune, J. L. Riordan, J. F. and Vallee, B. L. (1985) Amino acid sequence of human tumor derived angiogenin. Biochemistry 24, 5486.

Thrush, G. R., Lark, L. R. Clinchy, B. C. and Vietta, E. S. (1996) Immunotoxins: An Update Ann. Rev. Immunol, 14,49.

Youle, R. J. Newton, D. L., Wu, Y. N. Gadina, M. and Rybak, S. M. (1993) Cytoxic Ribonuclease and Shimeras in Cancer Therapy. Critical Reviews in Therapeutic Drug Carrier Systems 10,1.

Zewe, M. Rybak, S. M. Dubel, S., Coy, J. F. Welscholf, M., Newton, D. L. and Little M. (1997) Cloning and cytoxicity of human pancreatic RNase immunofusion. Immnotechnology 3, 127.

Other embodiments are within the following claims.

What is claimed is:

1. A method of making a transgenic fusion protein comprising providing a transgenic animal which includes a transgene which provides for the expression of the fusion protein; allowing the transgene to be expressed; and, recovering the fusion protein, from the milk of the transgenic animal.

2. The method of claim 1, wherein the fusion protein includes an immunoglobulin-subunit and an enzyme.

3. The method of claim 1, wherein the fusion protein includes a first member fused to a second member and the first member includes the subunit of a targeting molecule and the second member encodes a cell toxin.

4. The method of claim 1, wherein the fusion protein includes a subunit of an Ig specific for a tumor antigen.

5. The method of claim 4, wherein the tumor antigen is from the group carcinoembryonic antigen (CEA), a transferring receptor, TAG-72, an epidermal growth factor receptor.

6. The method of claim 1, wherein the fusion protein includes an Rnase.

7. The method of claim 6, wherein the RNase is RnaseA.

8. The method of claim 1, wherein the fusion protein includes angiogenin.

9. The method of claim 1, wherein the fusion protein includes carboxypeptidase B enzyme.

10. The method of claim 1, wherein the fusion protein is made in a mammary gland of the transgenic mammal.

11. The method of claim 1, wherein the fusion protein is secreted into the milk of a transgenic mammal at concentrations of at least about 0.5 mg/ml or higher.

12. The method of claim 1, wherein the fusion protein is secreted into the milk of a transgenic mammal at concentrations of at least about 1.0 mg/ml or higher.

13. The method of claim 1, the immunoglobulin subunit of a fusion protein is a humanized antibody.

14. The method of claim 1, wherein the transgene encoding the transgenic fusion protein is a nucleic acid construct which includes:
(a) optionally, an insulator sequence;
(b) a mammary epithelial specific promoter;
(c) a nucleotide sequence which encodes a signal sequence which can direct the secretion of the fusion protein, e.g. a signal from a milk specific protein;
(d) optionally, a nucleotide sequence which encodes a sufficient portion of the amino terminal coding region of a secreted protein, e.g. a protein secreted into milk, to allow secretion, e.g., in the milk of a transgenic mammal, of the fusion protein;
(e) one or more nucleotide sequences which encode the fusion protein; and
(f) optionally, a 3′ untranslated region from a mammalian gene.

15. An isolated nucleic acid construct, which includes:
(a) optionally, an insulator sequence;
(b) a mammary epithelial specific promoter;
(c) a nucleotide sequence which encodes a signal sequence which can direct the secretion of the fusion protein, e.g. a signal sequence from a milk specific protein;
(d) optionally, a nucleotide sequence which encodes a sufficient portion of the amino terminal coding region of a secreted protein, e.g. a protein secreted into milk, to allow secretion, e.g., in the milk of a transgenic mammal, of fusion protein;
(e) one or more nucleotide sequences which encode a fusion protein as described in claim 1; and
(f) optionally, a 3′ untranslated region from a mammalian gene, e.g., a mammary epithelial specific gene, (e.g., a milk protein gene).
In another aspect, the invention features, a pharmaceutical or nutraceutical composition having an effective amount of fusion protein, e.g., an immunoglobulin-enzyme fusion protein as described herein, and a pharmaceutically acceptable carrier.
In a preferred embodiment, the composition includes milk.

15. A transgenic animal which includes a transgene that encodes a fusion protein described in claim.

16. The transgenic animal of claim 15, which can secrete the fusion protein into its milk at concentrations of at least about 0.5 mg/mll or higher.


SEQUENCE LISTING (11 sequences)

SEQ ID NO: 1 - DNA, 705 bp, Artificial
  complete humanized light chain sequence
atggattttcaagtgcagattttcagcttcctgctaatcagtgcttcagtcataatgtcccgcggcgacatccagatgacccagagcccaagcagcctgagcgctagcgtgggtgacagagtgaccatcacgtgtagtgccagctcaagtgtaacttacatgcactggtaccagcagaagccaggtaaggctccaaagctgctgatctacagcacatccaacctggcttctggtgtgccaagcagattctccggaagcggtagcggcaccgactacaccttcaccatcagcagcctccagccagaggatatcgccacctactactgccagcagaggagtacttacccgctcacgttcggccaagggaccaagctcgagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 2 - protein, 235 aa, Artificial
  complete humanised light chain sequence
MDFQVQIFSFLLISASVIMSRGDIQMTQSPSSLSASVGDRVTITCSASSSVTYMHWYQQKPGKAPKLLIYSTSNLASGVPSRFSGSGSGTDYTFTISSLQPEDIATYYCQQRSTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1870 bp, Artificial
  expected PCR insert
aagcttgccgccaccatgaagttgtggctgaactggattttccttgtaacacttttaaatggaattcagtgtgaggtgcagctgcagcagagcggtccaggtctcgtacggcctagccagaccctgagcctcacgtgcaccgcatctggcttcaacattaaggacaattacatgcactgggtgagacagccacctggacgaggccttgagtggattggatggattgaccctgagaatggtgacactgagtacgcacctaagtttcgcggccgcgtgacaatgctggcagacactagtaagaaccagttcagcctgagactcagcagcgtgacagccgccgacaccgcggtctattattgtcacgtcctgatatacgccgggtatctggcaatggactactggggccaagggaccctcgtcaccgtgagctcggctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagtggagctgaaaaccccactcggtgacacaactcacacgtgccctaggtgtcctgaacctaaatcttgtgacacacctcccccgtgcccacggtgcccagagcccaaatcttgcgacacgcccccaccgtgtcccagatgtcctgaaccaaagagctgtgacactccaccgccctgcccgaggtgcccagcacctgaactcctgggaggagcaacaggacacagttatgagaagtacaacaagtgggaaacgatagaggcttggactcaacaagtcgccactgagaatccagccctcatctctcgcagtgttatcggaaccacatttgagggacgcgctatttacctcctgaaggttggcaaagctggacaaaataagcctgccattttcatggactgtggtttccatgccagagagtggatttctcctgcattctgccagtggtttgtaagagaggctgttcgtacctatggacgtgagatccaagtgacagagcttctcgacaagttagacttttatgtcctgcctgtgctcaatattgatggctacatctacacctggaccaagagccgattttggagaaagactcgctccacccatactggatctagctgcattggcacagaccccaacagaaattttgatgctggttggtgtgaaattggagcctctcgaaacccctgtgatgaaacttactgtggacctgccgcagagtctgaaaaggagaccaaggccctggctgatttcatccgcaacaaactctcttccatcaaggcatatctgacaatccactcgtactcccaaatgatgatctacccttactcatatgcttacaaactcggtgagaacaatgctgagttgaatgccctggctaaagctactgtgaaagaacttgcctcactgcacggcaccaagtacacatatggcccgggagctacaacaatctatccttctgctgggacttctaaagactgggcttatgaccaaggaatcagatattccttcacctttgaacttcgagatacaggcagatatggctttctccttccagaatcccagatccgggctacctgcgaggagaccttcctggcaatcaagtatgttgccagctacgtcctggaacacctgtactaataatctagagaga

SEQ ID NO: 4 - protein, 613 aa, Artificial [sequence omitted]

SEQ ID NO: 5 - protein, 96 aa, Artificial
  preproHCPB with C-terminal Leu
HHGGEHFEGEKVFRVNVEDENHINIIRELASTTQIDFWKPDSVTQIKPHSTVDFRVKAEDTVTVENVLKQNELQYKVLISNLRNVVEAQFDSRVRL

SEQ ID NO: 6 - protein, 40 aa, peptide linker
SGGGGSGGGGSGGGGSGGGGSGGGGSGGGGSGGGGSGGGG

SEQ ID NO: 7 - protein, 11 aa, peptide linker
GGGGSGGGGGS

SEQ ID NO: 8 - protein, 20 aa, peptide linker
SGGGGSGGGGSGGGGSGGGG

SEQ ID NO: 9 - protein, 17 aa, peptide linker
SSSSGSSSSGSSSSGSP

SEQ ID NO: 10 - DNA, 20 bp, nucleic acid primer
tgtgctcctctccatgctgg

SEQ ID NO: 11 - DNA, 20 bp, nucleic acid primer
tggtctggggtgacacatgt